SEQUENCE LISTING (32 sequences)

SEQ ID NO: 1 - protein, 25 aa, synthetic construct
ATACATGCAAGTCGAACGCTTCTTT

SEQ ID NO: 2 - protein, 25 aa, synthetic construct
CCACAAACCGTTCTACTTTACTGGC

SEQ ID NO: 3 - protein, 23 aa, synthetic construct
TCACCCTGTTTGATGGCCGCACC

SEQ ID NO: 4 - protein, 23 aa, synthetic construct
CTGAGCTTTGAATATCGCAGCCT

SEQ ID NO: 5 - protein, 19 aa, synthetic construct
CCGACCTCACAGCCCGAAA

SEQ ID NO: 6 - protein, 25 aa, synthetic construct
CGGTTCGCTTTCACTTTTCTTCAGC

SEQ ID NO: 7 - protein, 24 aa, synthetic construct
ACTGAGCTTTGAATATCGCAGCCT

SEQ ID NO: 8 - protein, 24 aa, synthetic construct
CGGTTCGCTTTCACTTTTCTCAGC

SEQ ID NO: 9 - protein, 25 aa, synthetic construct
ATACATGCAAGTCGAACGCTTCTTT

SEQ ID NO: 10 - protein, 25 aa, synthetic construct
CCACAAACCGTTCTACTTTACTGGC

SEQ ID NO: 11 - protein, 23 aa, synthetic construct
TCACCCTGTTTGATGGCCGCACC

SEQ ID NO: 12 - protein, 19 aa, synthetic construct
CCGACCTCACAGCCCGAAA

SEQ ID NO: 13 - protein, 19 aa, synthetic construct
AATGCGAACCAAATATGCC

SEQ ID NO: 14 - protein, 21 aa, synthetic construct
CAAGTTTTAGGACAGTCTGCT

SEQ ID NO: 15 - protein, 20 aa, synthetic construct
GGGATCGCATTAAATTACCT

SEQ ID NO: 16 - protein, 21 aa, synthetic construct
GCGATTGATGGTGATACGGTT

SEQ ID NO: 17 - protein, 19 aa, synthetic construct
GGGGGATCTTCGGACCTCA

SEQ ID NO: 18 - protein, 23 aa, synthetic construct
TGGTAATTACCGACGAAAACGGC

SEQ ID NO: 19 - protein, 22 aa, synthetic construct
CCGGAACTGGTTTCATCTGATT

SEQ ID NO: 20 - protein, 20 aa, synthetic construct
CTGAGCCAGGATCAAACTCT

SEQ ID NO: 21 - protein, 20 aa, synthetic construct
TCACCCTGTTTGATGGCCGC

SEQ ID NO: 22 - protein, 15 aa, synthetic construct
TCACCCTGTTTGATG

SEQ ID NO: 23 - protein, 10 aa, synthetic construct
TCACCCTGTT

SEQ ID NO: 24 - protein, 18 aa, synthetic construct
TTGCCAATATCGGCGGCC

SEQ ID NO: 25 - protein, 15 aa, synthetic construct
CCAATATCGGCGGCC

SEQ ID NO: 26 - protein, 15 aa, synthetic construct
AACTGGTTTCATCTG

SEQ ID NO: 27 - protein, 15 aa, synthetic construct
TCCGCAGTTTACGAC

SEQ ID NO: 28 - protein, 15 aa, synthetic construct
TTCGCAGTTTACGGC

SEQ ID NO: 29 - protein, 16 aa, synthetic construct
TCAATGAGCAAAGGTC

SEQ ID NO: 30 - protein, 18 aa, synthetic construct
GCCCTTTGTATTGTCCAC

SEQ ID NO: 31 - protein, 16 aa, synthetic construct
CACCTACACACCAGCC

SEQ ID NO: 32 - protein, 15 aa, synthetic construct
AACTTGCTGAACCAC

(19) United States
(12) Patent Application Publication
(10) Pub. No.: US 2025/0361569 A1
(43) Pub. Date: Nov. 27, 2025

(54) MULTIPLEXED PATHOGEN DETECTION USING NANOPLASMONIC SENSOR FOR URINARY TRACT INFECTIONS

(71) Applicant: Nanopath Inc., Cambridge, MA (US)

(72) Inventors: Alison Burklund, Cambridge, MA (US); Amogha Tadimety, Cambridge, MA (US); M. Nabuan Naufer, Billerica, MA (US)

(21) Appl. No.: 18/875,064

(22) PCT Filed: Jun. 15, 2023
(86) PCT No.: PCT/US2023/068547

§ 371 Date: Dec. 13, 2024

Related U.S. Application Data
(60) Provisional application No. 63/352,989, filed on Jun. 16, 2022.

(51) Int. Cl. C12Q 1/689; C12Q 1/6825; G16B 40/10

(52) U.S. Cl. CPC C12Q 1/689; C12Q 1/6825; G16B 40/10

(57) ABSTRACT

Disclosed herein includes a nanoplasmonic sensor for molecular characterization of urinary tract infections. In some embodiments, the nanoplasmonic sensor can also be used at the point-of-care. The nanoplasmonic sensor utilizes an optical phenomenon that occurs between a metal nanoparticle and a dielectric—localized surface plasmon resonance (LSPR)—for the detection of bacterial nucleic acids. In some embodiments, the spectral peak shift is a function of target sequence concentration.

### INCORPORATION BY REFERENCE TO ANY PRIORITY APPLICATIONS

This application claims priority to U.S. Provisional Patent Application No. 63/352,989 filed on Jun. 16, 2022. Any and all applications, if any, for which a foreign or domestic priority claim are hereby incorporated by reference in their entireties.

### REFERENCE TO SEQUENCE LISTING

The present application is being filed along with a Sequence Listing submitted electronically in XML format. The Sequence Listing is provided as a file entitled NPATH.007WOSEQLISTING.xml, created Jun. 15, 2023, which is approximately 29,065 bytes in size. The information in the electronic format of the Sequence Listing is incorporated herein by reference in its entirety.

### BACKGROUND

### Field of the Invention

This disclosure is related to the field of molecular detection. Specifically, the disclosure describes a method for functionalization of nanoplasmonic sensor and a functionalized nanoplasmonic sensor for the molecular characterization of urinary tract infections (UTIs).

### Description of the Related Art

Urinary tract infections (UTIs) are among the most common causes of a healthcare visit for women in the United States, with over 50% of women experiencing a UTI at some point in their life and represent one of largest sources of antibiotic prescriptions in the country. More specifically, UTIs have an annual prevalence of over 11% of the US population (>20% in elderly populations) with 15%-20% of those cases being resistant to first-line antibiotic therapy. Untreated UTIs can lead to severe complications for the patient, including systemic bacterial infections such as bacteremia. UTIs are caused by a wide range of pathogens, with the most common being Escherichia coli, Klebsiella pneumoniae, Proteus mirabilis, Enterococcus faecalis, and Staphylococcus saprophyticus. High recurrence rates associated with UTIs along with the increasing prevalence of antimicrobial resistant pathogens could result in a severe increase in healthcare costs and burden to healthcare system.

Despite the severity and prevalence of UTIs, diagnostic methodologies remain extremely time-consuming, and rely on antiquated culture-based methodologies for pathogen detection, followed by additional steps for species identification and characterization of antibiotic susceptibility. This time-intensive diagnostic workflow typically leaves women in pain for up to three days before they are prescribed the appropriate antibiotic therapy. In the most severe of cases, women are non-specifically prescribed empiric antibiotic therapy; however, due to the inability to prescribe targeted therapy, changes to treatment are needed in up to one third of cases. Furthermore, UTIs, especially those acquired in healthcare settings, are one of the major drivers of antibiotic resistance.

To the best of the Applicant's knowledge, there are no molecular diagnostics for UTIs and no existing technologies can identify UTI-causing pathogens at the point-of-care. Oftentimes, healthcare providers use urine dipsticks, which measure indirect markers of infection (e.g. pH), but suffer from lack of sensitivity and specificity. Other technologies employed in the bacterial characterization space are culture-based methods and nucleic acid amplification tests (NAATs). Some technologies that are commonly used for nucleic acid identification include quantitative polymerase chain reaction (qPCR), nucleic acid microarrays, amplicon-based metagenomic sequencing, and isothermal nucleic acid amplification tests (e.g. loop-mediated isothermal amplification, CRISPR-based assays, rolling circle amplification). That said, these molecular diagnostic technologies are not being utilized for the diagnosis and/or characterization of UTIs.

### SUMMARY

Disclosed herein is a nanoplasmonic sensor. In some embodiments, the nanoplasmonic sensor comprises: an array of functionalized sensors, wherein each of the functionalized sensors in the array comprises an array of nanostructures conjugated to a biological probe, and the biological probe is configured to detect the presence of a urinary tract infection-causing pathogen. In some embodiments, at least one of the functionalized sensors in the array comprises a different biological probe for detecting a different urinary tract infection-causing pathogen from the other functionalized sensors. In some embodiments, the nanoplasmonic sensor is configured to simultaneously detect multiple strands or species of the urinary tract infection-causing pathogens. In some embodiments, each of the functionalized sensors in the array comprises a different biological probe. In some embodiments, the urinary tract infection-causing pathogen is selected from the group consisting of Escherichia coli, Klebsiella pneumoniae, Proteus mirabilis, Enterococcus faecalis, Staphylococcus saprophyticus, and an antibiotic-resistant strand thereof. In some embodiments, the biological probe has a sequence selected from the group consisting of Seq. ID Nos. 1-32. In some embodiments, the nanostructures comprise gold. In some embodiments, the nanostructures in the array are regularly-spaced apart with a spacing of from about 100 nm and about 1000 nm, and each nanostructure has a square shape with a side dimension of from about 50 nm to about 400 nm. In some embodiments, the nanostructures have a thickness of from about 20 nm to about 75 nm.

Also disclosed herein is a method for detecting the presence of one or more urinary tract infection-causing pathogens. In some embodiments, the method comprises: (1) exposing the nanoplasmonic sensor of any of the embodiments disclosed herein to a bodily fluid sample of a patient suspecting of having urinary tract infection, (2) illuminating a light at a series of wavelengths onto each of the functionalized sensors, and (3) collecting absorbance, transmittance, or extinction data of each functionalized sensor. In some embodiments, the method further comprises comparing the collected absorbance, transmittance, or extinction data of each functionalized sensor with a baseline data of each of the functionalized sensor prior to exposure to the bodily fluid sample. In some embodiments, the comparing step reveals an optical peak shift when a urinary tract infection-causing pathogen is detected. In some embodiments, the amount of the optical peak shift is correlated to the concentration of the urinary tract infection-causing pathogen in the bodily fluid sample. In some embodiments, the bodily sample comprises urine. In some embodiments, at least one of the functionalized sensors in the array comprises a different biological probe for detecting a different urinary tract infection-causing pathogen from the other functionalized sensors. In some embodiments, the urinary tract infection-causing pathogen is independently selected from the group consisting of Escherichia coli, Klebsiella pneumoniae, Proteus mirabilis, Enterococcus faecalis, Staphylococcus saprophyticus, and an antibiotic-resistant strain or identified resistance gene thereof. In some embodiments, the biological probe is independently selected from the group consisting of Seq. ID Nos. 1-32. In some embodiments, each of the functionalized sensors in the array comprises a different biological probe. In some embodiments, multiple strands or species of the urinary tract infection-causing pathogens are detected simultaneously. In some embodiments, the method is configured to be performed at the point of care.

Another method for detecting the presence of one or more urinary tract infection-causing pathogens comprises providing a sensor comprising one or more biological probes designed to target specific nucleic acid sequences derived from one or more urinary tract infection-causing pathogens, exposing the sensor to a sample that is suspected to contain one or more urinary tract infection-causing pathogens, and collecting electrical, fluorescent, absorbance, transmittance, and/or extinction data from the sensor. In some embodiments, the one or more biological probes were selected using computational and/or bioinformatic methods. In some embodiments, the one or more biological probes contain intentionally varying degrees of mismatch with the target nucleic acids. In some embodiments, the one or more biological probes are designed to bind multiple target nucleic acid sequences. In some embodiments, one of the biological probes can bind nucleic acids derived from more than one urinary tract infection-causing pathogen. In some embodiments, the one or more biological probes are designed to bind nucleic acid sequences specific to antibiotic resistance genes. In some embodiments, one of the biological probes can bind nucleic acid sequences from more than one antibiotic resistance genes.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A depict one embodiment of a plasmonic-resonance sensing device.

FIG. 1B depicts one embodiment of an array of nanostructures in a sensor of the plasmonic-resonance sensing device.

FIGS. 2A-2B depict non-limiting example schematics of selected geometries and fabrication maps. FIG. 1A illustrates a schematic of a grid with labeled dimensions for length, width, thickness, and periodicity of nanostructures. FIG. 1B illustrates a schematic of a map of arrangement of dimensions for dose matrix test.

FIG. 3 shows extinction curves of a non-limiting example of regular gold nanorod array at three bulk refractive indices.

FIGS. 4A-4B depict examples of PNA-DNA Binding Simulations. The simulations are of conformal layers representing PNA and DNA binding to gold nanostructure. The two geometries demonstrated here are (FIG. 3A) repeating nanorod array (130 nm×40 nm) and (FIG. 3B) repeating nanosquare array (95 nm×95 nm).

FIG. 5A shows the extinction curve for bulk refractive index sensitivity simulations in one embodiment of the nanostructure arrays at three refractive indexes. FIG. 5B depicts the refractive index sensitivities of uncoupled nanorods and the nanostructure array.

FIG. 6A shows the extinction curve for bulk refractive index sensitivity simulations in one embodiment of the nanostructure arrays at three refractive indexes. FIG. 6B depicts the refractive index sensitivities of uncoupled nanorods and the nanostructure array.

FIG. 7A shows the extinction curve for bulk refractive index sensitivity simulations in one embodiment of the nanostructure arrays at three refractive indexes. FIG. 7B depicts the refractive index sensitivities of uncoupled nanorods and the nanostructure array.

FIG. 8 depicts the experimental transmission spectra for 5 different nanoarray geometries.

FIG. 9 depicts the simulated transmission spectra for each 5 different nanoarray geometries.

FIG. 10 depicts a CAD drawing of post array polymer well mold and fabricated well, with coordinates aligned over the sensor array.

FIGS. 11A-11B depict two views of an embodiment of 3D printed mold for a fabricated polymer well.

FIGS. 11C-11D depict two views of one embodiment of a fabricated well array, made from the mold shown in FIGS. 11A and 11B.

FIGS. 11E-11I depict additional embodiments of micro-well fixtures.

FIGS. 12A-12C depict one embodiment of the automatic pipette system. FIG. 12A depicts the overall system, with pipette holder on the left, tip box, 96-well plate holder, and custom chip adapter. FIG. 12B depicts the tip box aligned under pipette holder. FIG. 12C depicts the 96 well plate and adapter during functionalization.

FIGS. 13A-13C depict the Nanoplasmonic detection of target bacterial species in PBS and synthetic urine. Each biological replicate (n=3) was measured on three unique sensing spots. FIGS. 13A, 13B, and 13C each represent one of three measurements (technical replicates) that were taken on each sensing spot.

FIG. 14 depicts probe specificity analysis. The “channel” describes the PNA probe designed for species-level organism detection or detection of antimicrobial resistance genes, and “spike” describes the genetic material exposed to the sensor.

FIGS. 15A-15E depict the Nanosensor limit-of-detection for five targets in synthetic urine matrix. Each replicate (n=3) was measured on three unique sensing spots. Three measurements (technical replicates) were taken on each sensing spot. Dashed red line represents the upper bound of 95% confidence interval of negative control samples. FIG. 15A depicts the limit-of-detection for Channel: E. coli and Isolate: E. coli. FIG. 15B depicts the limit-of-detection for Channel: Enterococcus and Isolate: E. faecalis. FIG. 15C depicts the limit-of-detection for Channel: K. pneumoniae and Isolate: K. pneumoniae. FIG. 15D depicts the limit-of-detection for Channel: CT-X-M1 and Isolate: E. coli with bla_{CTX-M-1}. FIG. 15E depicts the limit-of-detection for Channel: VanA and Isolate: E. faecalis with VanA.

FIGS. 16A-16E depict the evaluation of nanosenor performance in healthy patient urine sample matrix. Each circle represents an individual patient urine matrix. Diamonds represent pooled-patient urine matrix (if applicable). Dashed red line represents the upper bound of 95% CI of the negative control samples. FIG. 16A depicts the shift in detection for Channel: E. coli and Isolate: E. coli. FIG. 16B depicts the shift in detection for Channel: Enterococcus and Isolate: E. faecalis. FIG. 16C depicts the shift in detection for Channel: K. pneumoniae and Isolate: K. pneumoniae. FIG. 16D depicts the shift in detection for Channel: CT-X-M1 and Isolate: E. coli with bla_{CTX-M-1}. FIG. 16E depicts the shift in detection for Channel: VanA and Isolate: E. faecalis with VanA.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

A plasmon-resonance sensing device employing ordered array nanostructure ensembles is described herein. The ordered array of nanostructures allows for coupling to diffractive photonic modes, which can be used to improve sensor sensitivity. The nanostructure dimension and geometry are tailored to provide high quality signal and large optical shifts upon modeled analyte binding.

The present disclosure generally relates to a nanoplasmonic biosensor for point-of-care molecular characterization of urinary tract infections. The technology of the present disclosure employs an optical phenomenon that occurs between a metal nanoparticle and a dielectric—localized surface plasmon resonance (LSPR)—for the detection of pathogen nucleic acids. LSPR is observed when the wavelength of incident light is larger than the size of the conductive nanoparticles and presents an opportunity for highly sensitive detection of specific nucleic acid sequences. In this disclosure, nanostructures are covalently functionalized with biological probes. The nanostructures result in highly confined electric fields of LSPR modes, which serve as a sensitive transducer to changes in the local dielectric environment (i.e., a binding event). In some embodiments, upon hybridization to the nucleic acid target sequences, successive red shifts in the spectral peak as a function of target sequence concentration can be observed. The ordered array of nanostructures allows for coupling to diffractive photonic modes, which can lead to improved sensor sensitivity. The nanostructure dimension and geometry are tailored to provide high quality signal and large optical shifts upon modeled analyte binding.

Also disclosed herein is a nanoplasmonic sensor for rapid (<15 min) molecular characterization of urinary tract infections. The nanoplasmonic sensor of the present disclosure harnesses an optical phenomenon that occurs between a metal nanoparticle and a dielectric—localized surface plasmon resonance (LSPR)—for the detection of bacterial nucleic acids. The sensing substrate is functionalized with rationally designed biological probes (PNAs) that are complementary to DNA targets of interest. The panel described herein identifies genetic sequences specific to Escherichia coli, Enterococcus spp., Klebsiella pneumoniae, vancomycin-resistance (vanA), vancomycin-resistance (vanA/B), and extended-spectrum beta-lactamase producers (CTX-M). For these targets, there is a significant red-shift in peak absorbance wavelength of the sensor when target DNA was exposed to the functionalized nanosensing substrate, suggesting successful hybridization of the target nucleic acid sequence to the complementary biological probe. Probes were observed to be highly specific to their target of interest and there was no significant cross-reactively. For all the targets, a significant peak wavelength shift was first observed at a cell load (or equivalent) of approximately 10⁴ CFU/mL. The magnitude of the peak wavelength shift (i.e. signal) successively increased with increasing target concentration, suggesting the feasibility of semi-quantitative sample characterization, which is advantageous for the clinical management of UTIs. Lastly, real patient urine sample matrices (n=5) had no significant effect on the nanoplasmonic sensor performance. These results suggest that this platform can rule-in clinically significant UTIs, identify the UTI-causing organism, and characterize key antimicrobial resistance profiles within 15 minutes. This technology platform is enabling the first DNA-based test for UTI diagnosis and characterization at the point-of-care.

### Plasmon-Resonance Sensing Devices

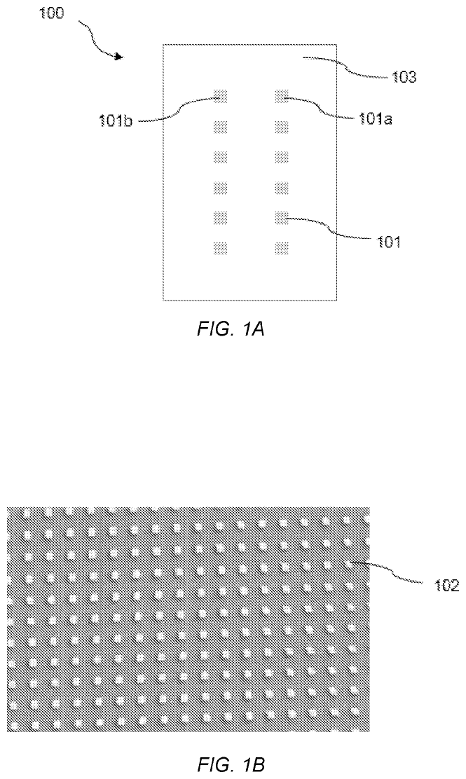

Disclosed herein is a plasmon-resonance sensing device. As shown in FIGS. 1A and 1B, the plasmon-resonance sensing device 100 comprises an array of sensors 101. Each sensor 101 comprises an array of nanostructures 102 that are regularly spaced apart. In some embodiments, the nanostructures 102 are regularly spaced apart with a spacing of about 100 nm, about 200 nm, about 300 nm, about 500 nm, about 750 nm, about 1000 nm, about 1200 nm, about 1500 nm, about 1800 nm, about 2000 nm, or any distance that is between about 100 nm and about 2000 nm, between the nanostructures. In some embodiments, the array of nanostructures are regularly spaced apart with a spacing of from about 100 nm to about 2000 nm, from about 100 nm to about 1800 nm, from about 100 nm to about 1600 nm, from about 100 nm to about 1400 nm, from about 100 nm to about 1200 nm, from about 100 nm to about 1000 nm, from about 200 nm to about 900 nm, from about 300 nm to about 800 nm, from about 100 nm to about 400 nm, from about 200 nm to about 500 nm, from about 300 nm to about 600 nm, from about 400 nm to about 700 nm, from about 500nm to about 800 nm, from about 600 nm to about 900 nm, from about 700 nm to about 1000 nm, from about 500 nm to about 2000 nm, or from about 500 nm to about 1500 nm between the nanostructures.

The nanostructures in the array may have various shapes. For example, the nanostructures may have a rectangular shape, a circular shape, a triangular shape, a star shape, a pentagon shape, a parallelogram shape, a diamond shape, or a square shape. Preferably, each of the nanostructures in the array has a square shape. In some embodiments, each nanostructure has a side dimension of about 50 nm, about 75 nm, about 100 nm, about 150 nm, about 200 nm, about 250 nm, about 300 nm, about 350 nm, or about 400 nm, or any integer that is between about 50 to about 400 nm. In some embodiments, the square shape has a side dimension of from about 50 nm to about 400 nm, from about 100 nm to about 350 nm, from 150 nm to about 300 nm, from about 50 nm to about 150 nm, from about 100 nm to about 200 nm, from 150 nm to about 250 nm, from about 200 nm to about 300 nm, from about 250 nm to about 350 nm, or from about 300 nm to about 400 nm, or any range that is between about 50 nm and about 400 nm.

In some embodiments, the nanostructures in the array may have a thickness of about 20 nm, about 25 nm, about 30 nm, about 35 nm, about 40 nm, about 45 nm, about 50 nm, about 60 nm, about 65 nm, about 70 nm, about 75 nm, or any integer between about 20 and about 75 nm. In some embodiments, the nanostructures in the array may have a thickness of from about 20 nm to about 75 nm, from about 25 nm to about 70 nm, from about 30 nm to about 65 nm, from about 35 nm to about 60 nm, from about 30 nm to about 55 nm, or any range that is between about 20 and about 75 nm.

The nanostructures comprise a metal. For example, the nanostructures may comprise gold, platinum, aluminum, silver, or copper. Preferably, the nanostructure comprises gold. In some embodiments, the nanostructures comprise a single metal. In some embodiments, the nanostructures comprise a mixture of metals.

In some embodiments, the nanostructures in the array are conjugated with a biological probe. The biological probe is configured to bind to an analyte. The binding of the analyte to the biological probe alters the surface properties of the nanostructure, thereby causing a change in localized surface plasmon resonance. In some embodiments, the biological probe comprises one or more of a protein, peptide strand, amino acid, RNA strand, DNA strand, or and/or nucleotide. In some embodiments, the biological probe comprises one or more of a modified protein, modified peptide, modified amino acid, modified RNA strand, modified DNA strand, and/or modified nucleotide. In some embodiments, the biological probe comprises at least one of: a peptide-nucleic acid, an aptamer, an antibody, an antibody fragment, a complimentary DNA, and/or an enzyme. In some embodiments, the biological probe is selected from the group consisting of a peptide-nucleic acid, an aptamer, an antibody, an antibody fragment, a complimentary DNA, and an enzyme.

In some embodiments, at least a first sensor 101a in the array of sensors comprises nanostructures 102 conjugated with a first biological probe. In some embodiments, at least a second sensor 101b in the array of sensors comprises nanostructures conjugated with a second biological probe. In some embodiments, at least a third sensor in the array of sensors comprises nanostructures conjugated with a third biological probe. In some embodiments, at least a fourth sensor in the array of sensors comprises nanostructures conjugated with a fourth biological probe. In some embodiments, at least a fifth sensor in the array of sensors comprises nanostructures conjugated with a fifth biological probe. In some embodiments, a “n” number of sensors in an array of sensors comprises nanostructures conjugated to an “n” number of biological probes, wherein “n” is any number from 1 to 2000 In some embodiments, 6 or 12 sensors may be presented in the array of sensors on a substrate 103. In some embodiments, the sensors may have an area of from about 1 μm² to about 1 mm². In some embodiments, the sensors may have an area of from about 10 μm² to about 1 mm², about 50 μm² to about 1 mm², about 100 μm² to about 1 mm², about 200 μm² to about 1 mm², about 400 μm² to about 1 mm², or about 500 μm² to about 1 mm².

The substrate 103 may be a dielectric or non-conductive substrate. In some embodiments, the substrate 103 is transparent to allow the sensors to be exposed to the incident light through the substrate 103. For example, the substrate 103 may be a glass, a plastic, or a polymeric substrate. In some embodiments, the substrate 103 may be a polymer substrate or a plastic substrate. The substrate and the sensor array on the substrate may be integrated with a microfluidic module to provide a means for introducing or exposing the sample to the sensors.

### Analyte Detection

Disclosed herein is a method for detecting an analyte in a sample. In some embodiments, the method comprises exposing at least one sensor 101 in the plasmon-resonance sensing device 100 of any of the embodiments disclosed herein to a sample. The sample may or may not comprise the target analyte. The plasmon-resonance sensing device 100 can be utilized to detect the presence of an analyte (i.e., a target analyte). In some embodiments, the method comprises exposing at least two sensors in the plasmon-resonance sensing device 100 of any of the embodiments disclosed herein to a sample. In some embodiments, the method comprises exposing at least three sensors, at least four sensors, at least 5 sensors, or at least 6 sensors in the plasmon-resonance sensing device 100 of any of the embodiments disclosed herein to a sample. In some embodiments, the method comprises exposing an “n” number of sensors in the plasmon-resonance sensing device of any of the embodiments disclosed herein to a sample, wherein “n” is any number from 1 to 2000. In some embodiments, the array of sensors is exposed to the sample. The sample may comprise a bodily fluid, such as blood, plasma, mucus, serum, urine, or saliva, etc. Mucus can be collected via cervical swabs, vaginal swabs, or nasal swabs. When the at least one sensor 101 is exposed to the sample, the biological probe in each sensor would selectively bind to the analyte that the biological probe is configured to bine.

Optionally, the at least one sensor may be subject to a heating step after the exposure to the sample. In some embodiments, the at least one sensor is heated up to about 85° C. or any temperature between 25° C. and 85° C. In some embodiments, the at least one sensor may be exposed to heat before, during, or after subsequent steps. In some embodiments, the at least one sensor may be exposed to heat before, during, or after the measurement.

The method for detecting or sensing an analyte further comprises illuminating a light onto the at least one sensor. In some embodiments, the method comprises illuminating a light at a series of wavelengths onto the at least one sensor. In some embodiments, the light may be emitted from a light source in an apparatus for analyte detection. The light source may be configured to emit a series of wavelengths for illuminating the sensor. In some embodiments, the plasmonic sensing chip containing the sensors may be inserted into the apparatus for analyte detection. The apparatus is configured to emit a light at a series of wavelengths onto the sensors, and to collect an optical spectrum of the light transmitted through, absorbed by, or reflected from the sensors. For example, the apparatus can perform absorbance/transmittance measurements. In some embodiments the measurements are made at wavelengths ranging from 500-1000 nm.

The method further comprises collecting data from the sensor. In some embodiments, the method comprises collecting absorbance data from the sensor. In some embodiments, the method comprises collecting transmittance data from the sensor. In some embodiments, the method comprises collecting extinction data from the sensor. In some embodiments, the method comprises collecting absorbance, transmittance, and/or extinction data of the sensor. In some embodiments, the method further comprises comparing collected data with a baseline data of the sensor prior to the sample exposure. In some embodiments, the method further comprises comparing at least one of the collected absorbance, transmittance, and/or extinction data with a baseline data of the sensor prior to the sample exposure. For example, the absorbance/transmittance measurements of functionalized sensors are made prior to exposure to the sample. The peak absorbance wavelength of the functionalized sensor (prior to bonding with a target analyte) is identified. The absorbance/transmittance of the sensors are made again after exposing to the sample, and a shift in peak absorbance can be observed if a target analyte is present in the sample and binds with the probe on the functionalized sensors. The shift represents the detection signal.

In some embodiments, an array of sensors in the plasmon-resonance sensing device 100 of any of the present embodiments is exposed to the sample. In some embodiments, at least a first sensor 101a in the array of sensors 101 comprises nanostructures conjugated with a first biological probe. In some embodiments, at least a second sensor 101b in the array of sensors 101 comprises nanostructures conjugated with a second biological probe. In some embodiments, at least a third sensor in the array of sensors comprises nanostructures conjugated with a third biological probe. In some embodiments, at least a fourth sensor in the array of sensors comprises nanostructures conjugated with a fourth biological probe. In some embodiments, at least a fifth sensor in the array of sensors comprises nanostructures conjugated with a fifth biological probe. In some embodiments, a “n” number of sensors in an array of sensors comprises nanostructures conjugated to an “n” number of biological probes, wherein “n” is any number from 1 to 2000. The biological probes conjugated to different sensors may be the same or different. In some embodiments, each sensor in the array can be conjugated to different biological probes for a multiplex sensing capability. In this configuration, multiple analytes can be detected simultaneously.

In some embodiments, at least a first sensor 101a in the array of sensors comprises nanostructures conjugated with a first biological probe and at least a second sensor 101b in the array of sensors comprises nanostructures conjugated with a second biological probe. In some embodiments, a first set of sensors in the sensor array is functionalized with a first biological probe, and a second set of sensors in the sensor array is functionalized with a second biological probe. In some embodiments, the first biological probe and the second biological probe are different. In some embodiments, the first biological probe and the second biological probe are the same. In some embodiments, the first biological probe and the second biological probe independently comprise one or more of a protein, peptide strand, amino acid, RNA strand, DNA strand, or and/or nucleotide. In some embodiments, the first biological probe and the second biological probe independently comprise one or more of a modified protein, modified peptide, modified amino acid, modified RNA strand, modified DNA strand, and/or modified nucleotide. In some embodiments, the first biological probe and the second biological probe independently comprise at least one of: a peptide-nucleic acid, an aptamer, an antibody, an antibody fragment, a complimentary DNA, and/or an enzyme. In some embodiments, first biological probe and the second biological probe are independently selected from the group consisting of a peptide-nucleic acid, an aptamer, an antibody, an antibody fragment, a complimentary DNA, and an enzyme.

The detection of analyte(s) is based on an optical phenomenon that occurs between a metal nanostructure and a dielectric—localized surface plasmon resonance (LSPR). LSPR is observed when the wavelength of incident light is larger than the size of the conductive nanostructures. The nanostructures result in highly confined electric fields of LSPR modes, which serve as a sensitive transducer to changes in the local dielectric environment (binding event). The nanostructures can be conjugated to/covalently functionalized with probes that can bind with target analytes. Upon binding with the target analyte(s), red shifts in the spectral peak can be observed. In some embodiments the amount of red shift may be observed as a function of target analyte concentration. In some embodiments, the sensors detect transmittance, reflectance, and/or absorbance at certain wavelength range.

In some embodiments, the sensors that have been exposed to the sample, thus having analyte(s) bound to selective biological probe on the sensors, can be further exposed to functionalized particles configured to bind to the sensors that have analyte(s) present and bound to the biological probe. The functionalize particles may be nanoparticles or microparticles. In some embodiments, the particles may be metal, polymer, glass, or any material with a high refractive index, for example, a refractive index of about 1.5 and higher. When the functionalized particles are bound to the sensors, it has the potential to improve both sensitivity and specificity of the sensors. Without being bound to the theory, the sensitivity improvements may be due to the fact that the functionalized particle increases the change in refractive index at the sensor surface in the presence of the analyte. The additional binding of the functionalized particles to the sensors may improve the sensor signal through a greater peak-shift in the optical measurement. Specificity improvements may be due to the fact that two selective binding events are required (i.e., first analyte must bind to the sensor, then the functionalized particle must bind to the sensor-bound analyte). In some embodiments, the functionalized particles are functionalized to bind to the analytes that have bound to the biological probes.

In some embodiments, a spectrum of the sensor comprising an array of functionalized nanostructures may be obtained prior to exposure to a sample. This may provide baseline data for the determination and analysis of an analyte binding event.

### Nanostructures Fabrication

Also disclosed herein is a method of making an array of nanostructures. The method comprises coating a photoresist layer onto a substrate, patterning the photoresist, and depositing a metallic layer over the patterned photoresist layer. In some embodiments, the substrate may be non-conductive, and a modified method may provide an improved result. The method comprises coating a conductive photoresist layer onto a non-conductive substrate, patterning the conductive photoresist layer via photolithography, depositing an adhesion layer over the patterned conductive photoresist layer, and depositing a metallic layer onto the adhesion layer. In some embodiments, patterning the conductive photoresist layer comprises exposing the photoresist layer to the electron beam to create a desired pattern. In some embodiments, the pattern should match the dimensions of and the spacing between the nanostructures. In some embodiments, the method may involve lithographic techniques, such as electron-beam lithography, UV photolithography, or nanoimprint lithography. In some embodiments, roll-to-roll manufacturing may be employed for making the sensor array.

For example, photolithography may be utilized to remove the portions of the photoresist layer where the nanostructures should be disposed/formed on the substrate, leaving the portion of the substrate where there should not be any nanostructure masked by the patterned photoresist layer. The patterned photoresist layer therefore has removed portions resembling the size, shape, and location of where the metallic nanostructures should be disposed. The portion of substrate is exposed at where the nanostructures will be formed. When the metallic layer is subsequently disposed over the patterned photoresist layer, some metallic layer would be disposed on the exposed portions of the substrate, and some the metallic layer would be disposed on the remaining photoresist that is masking the substrate.

The method further comprises lifting off the patterned photoresist layer. Lifting off the patterned photoresist layer also takes off the portions of the adhesive layer and the metallic layer disposed on the remaining patterned photoresist layer, leaving behind the portions of the adhesive layer that are in contact with the substrate and the portions of the metallic layer on that portions of the adhesive layer. In some embodiments, the adhesion layer comprises chromium. In some embodiments, the adhesion layer has a thickness of about 2 nm, about 3 nm, about 4 nm, about 5 nm, about 6 nm, about 8 nm, about 9 nm, or any thickness that is between about 2 and about 9 nm. In some embodiments, the adhesion layer has a thickness of about 5 nm. In some embodiments, the metallic layer comprises a single metal. In some embodiments, the metallic layer comprises a mixture of metals. In some embodiments, the metallic layer comprises gold, silver, aluminum, platinum or copper. In some embodiments, the metallic layer comprises gold. The thickness of the metallic layer would be the same as the thickness of the nanostructures on the substrate as disclosed herein.

The method disclosed herein provides an array of sensors comprising an array of nanostructures that are regularly spaced apart. The shape, dimensions, and the spacing of the nanostructures made by such method are the same as disclosed herein.

### Functionalization of Nanoplasmonic Sensing Chip

Disclosed herein is a method of making a functionalized nanoplasmonic sensing chip. The method comprises providing a substrate comprising an array of sensors, affixing a micro-well adaptor on top of the substrate so an array of micro-wells is over the array of sensors and aligned with each sensor, and forming one or more functionalized sensors in the array of sensors. Forming the one or more functionalized sensors includes delivering a first batch of reaction solutions into one or more micro-wells atop one or more sensors using an automatic pipetting system, and then subsequently removing the first batch of reaction solution from the one or more micro-wells using the automatic pipetting system. The automatic pipetting system includes an array of pipets that can be loaded with one or more reaction solutions. In some embodiments, the array of pipets may be loaded with two or more different reaction solutions, thus allowing delivery of two or more different reaction solutions to the array of micro-wells/sensors. The array of pipets may also be used to remove the reaction solutions from some or all of the micro-wells/sensors after the reactions. The array of pipets can deliver or remove reaction solutions from a specific micro-well/sensor or a specific group of micro-wells/sensors. In some embodiments, each reaction solution may include one or more reagents for modifying the array of nanostructures in the sensor. In some embodiments, each reaction solution may include one or more biological probes.

In some embodiments, multi-step reactions may be utilized for functionalizing the sensors. Thus, forming one or more functionalized sensors may further involve delivering a second batch of reaction solutions into the one or more micro-wells, and subsequently removing the second batch of reaction solutions from the one or more micro-wells, wherein the delivering and removing the second batch of reaction solutions are performed by an automatic pipetting system.

In some embodiments, the first batch of reaction solutions comprises two or more different reaction solutions. In some embodiments, the second batch of reaction solutions may also comprise two or more different reaction solutions. In some embodiments, the reaction solutions may include different biological probes. Thus the array of functionalized sensors may comprise two or more different biological probes. For example, some of the functionalized sensors in the array may comprise a specific biological probe, while other functionalized sensors comprise a different biological probe. In some embodiments, each of the functionalized sensor may comprise different biological probes. In some embodiments, a reaction solution may include one or more biological sensors. Thus each functionalized sensor may comprise one or more biological probes. One or more biological probes can conjugate to the array of nanostructures in each sensor. In some embodiments, the sensor may comprise one, two, three, four, or more biological probes configured to bind to one or more analytes.

Then the method further includes removing the micro-well adaptor from the substrate. In some embodiments, the one or more sensors are functionalized with a biological probe while the first batch of reaction solutions in the one or more micro-wells is in contact with the sensors. In some embodiments, the one or more sensors is functionalized with a biological probe after two or more reaction steps. In some embodiments, the sensor (e.g., the one or more sensors) each comprises an array of nanostructures disclosed herein.

In some embodiments, the automatic pipetting system can be configured to deliver different reaction solutions to multiple micro-wells for functionalizing multiple sensors in the array. In some embodiments, multiple reaction solutions are delivered to different sensors in the array, thereby functionalizing multiple sensors substantially at the same time. In some embodiments, the automatic pipetting system can be configured to removing different reaction solutions from multiple micro-wells. In some embodiments, multiple reaction solutions are removed from different sensors in the array substantially at the same time. In other embodiments, some reaction solutions may be removed at a different time to allow longer or shorter reaction time.

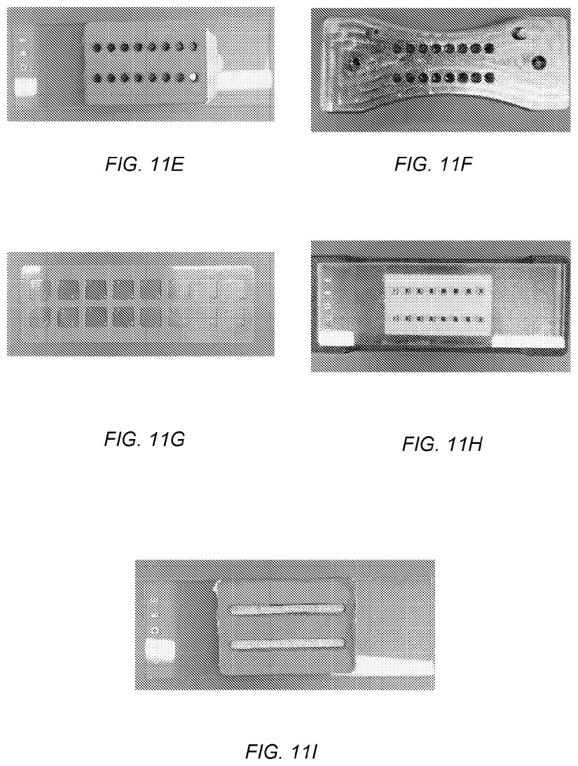

FIGS. 11A-11B depict two alternative views of a 3D printed mold for a fabricated polymer well shown in FIGS. 11C-11D. Other embodiments of the micro-wells are shown in FIGS. 11E-11I.

In some embodiments, additional pre-treatment step(s) can be performed prior to delivering any reaction solution. The pre-treatment step may include washing the nanostructure surface, wetting the nanostructure surface, or activation the nanostructure for subsequent reaction/functionalization. In some embodiments, the method may further comprise delivering an activation solution into at least a portion of the micro-wells atop the sensors in the array using an automatic pipetting system; and subsequently removing the activation solution prior to delivering a reaction solution.

The method disclosed herein provides at least one functionalized sensor comprises an at least one biological probe. In some alternatives, the first functionalized sensor comprises a first array of nanostructures conjugated to a first biological probe. In some alternatives, the second functionalized sensor comprises a second array of nanostructures conjugated to a second biological probe. In some alternatives, additional sensors comprising a nanostructures array may be conjugated to additional biological probe(s), up to the number of sensors in the sensor array. For example, a “n” number of sensors in an array of sensors comprises nanostructures conjugated to an “n” number of biological probes, wherein “n” is any number from 1 to 2000. In some embodiments, n may be any number from 1 to 1000, from 1 to 500, from 1 to 100, or from 1 to 25.

Each of the biological probes is independently selected from the group consisting of a peptide-nucleic acid (PNA), an oligonucleotide, an aptamer, an antibody, an antibody fragment, a complimentary DNA, and an enzyme. In some alternatives, the first biological probe and the second biological probe are independently selected from the group consisting of a peptide-nucleic acid, an aptamer, an antibody, an antibody fragment, a complimentary DNA, and an enzyme. In some alternatives, the first biological probe and the second biological probe are different. In some alternatives, the first biological probe and the second biological probe are the same. In some embodiments, each sensor may be functionalized with a different biological probe. In some embodiments, some of the sensors in the array may be functionalized with different biological probes. In some embodiments, all the sensors in the array may be functionalized with the same biological probe.

In some embodiments, reaction solutions are delivered to all the micro-wells simultaneously. In some alternatives, reaction solutions are subsequently removed from the micro-wells simultaneously. In some alternatives, reaction solutions are removed from the micro-wells at a different time to accommodate for different reaction time for functionalizing the sensors with a variety of the biological probes. In some embodiments, reaction solutions can also be delivered to different micro-wells at a different time. In some alternatives, the first reaction solution and the second reaction solution are delivered to the first micro-well and the second micro-well simultaneously, and subsequently the first reaction solution and the second reaction solution are removed from the first micro-well and the second micro-well. In some embodiments, delivering and removing a reaction solution may be performed by an automatic pipetting system. In some embodiments, the automatic pipetting system may be configured to remove different reaction solutions at a different time. In some embodiments, the automatic pipetting system may be configured to deliver different reaction solution at a different time.

In some embodiments, the nanostructures comprise a metal. In some alternatives, the nanostructures comprise a single metal. In some alternatives, the nanostructures comprise a mixture of metals. In some alternatives, the nanostructures comprise gold, platinum, aluminum, silver, or copper. In some alternatives, the nanostructures comprise gold.

### Functionalized Plasmonic Sensing Chip

Functionalized plasmonic sensing chips comprising an array of functionalized sensors are disclosed. In some embodiments, each of the functionalized sensors in the array comprises an array of nanostructures conjugated to at least one biological probe. In some embodiments, the array of nanostructures is conjugated to two or more biological probes configured to bind to two or more analytes. The biological probe is configured to bind to at least one analyte. In some alternatives, the at least one biological probe independently comprises at least one of: a peptide-nucleic acid, an oligonucleotide, an aptamer, an antibody, an antibody fragment, a complimentary DNA, and/or an enzyme. In some embodiments, the biological probe is independently selected from the group consisting of a peptide-nucleic acid, an oligonucleotide, an aptamer, an antibody, an antibody fragment, a complimentary DNA, and an enzyme. In some embodiments, all functionalized sensors in the array comprise the same biological probes. In some alternatives, at least one of the functionalized sensors in the array comprises at least one different biological probe from the others. For example, some of the functionalized sensors in the array may comprise a specific biological probe, while other functionalized sensors comprise a different biological probe. In some embodiments, each of the functionalized sensors in the array comprise at least one different biological probe. One or more biological probes can conjugate to the array of nanostructures in each sensor. In some embodiments, the functionalized sensor may comprise one, two, three, four, or more biological probes configured to bind to one or more analytes.

In some embodiments, the functionalized plasmonic sensor chip may include 1 to 100 (and any numbers in between) different biological probes. For example, the functionalized plasmonic sensor chip may include 1, 2, 3, 4, 5, 6, 7, 8, 9, 10, 11, 12, 13, 14, 15, 20, 24, 30, 36, 40, 48, 50, 54, 60, 70, 80, 90, or 100 different biological probes. In some embodiments, each functionalized sensor in the functionalized plasmonic sensor chip may contain different biological probe(s). In some embodiments, the array of the nanostructures in each sensor may conjugate to one or more biological probes, and the one or more biological probes may be different.

In some embodiments, the nanostructures comprise a metal. In some alternatives, the nanostructures comprise a single metal. In some alternatives, the nanostructures comprise a mixture of metals. In some alternatives, the nanostructures may comprise gold, platinum, aluminum, silver, or copper. In some alternatives, the nanostructures comprise gold. In some alternatives, the nanostructures in the array are regularly spaced apart and may have the geometry described herein.

### Multiplex Analyte Detections

A method for detecting two or more analytes simultaneously is also described. In some alternatives, the method may detect 3, 4, 5, 6, 7, 8, 9, 10, 11, 12, 13, 14, 15, 20, 24, 30, 36, 40, 48, 50, 54, 60, 70, 80, 90, and/or 100 analytes. In some embodiments, up to 50 analytes are detected. In some embodiments, up to 24, up to 50, up to 80, or up to 100 analytes may be detected. The method comprises exposing the array of functionalized sensors on the plasmonic sensing chip of any of the alternatives disclosed herein to a sample. The functionalized sensors are configured to detect the presence of certain target analytes. In some embodiments, the functionalized sensor may be configured to identify or detect various markers, subtypes, strains, genotypes and/or variants of a biological species. When the functionalized sensors are exposed to the sample, one or more target analytes, if present, bind to the corresponding biological probes. The binding event causes a change in the local dielectric environment of the sensors. The sample may comprise a bodily fluid, such as blood, urine, or saliva, etc. In some embodiments, the sample may be evacuated or removed from the functionalized sensors following the exposure step.

Optionally, the array of functionalized sensors may be subject to a heating step after the exposure to the sample. In some embodiments, the array of functionalized sensors is heated up to about 85° C. or any temperature between 25° C. and 85° C. In some embodiments, the array of functionalized sensors may be exposed to heat before, during, or after subsequent steps. In some embodiments, the array of functionalized sensors may be exposed to heat before, during, or after the measurement.

The method further comprises illuminating a light at a series of wavelengths onto the functionalized sensors; and collecting absorbance, transmittance, and/or extinction data from the functionalized sensors. The light may be emitted from a light source in an apparatus for analyte detection. The light source may be configured to emit a series of wavelengths for illuminating the sensors. In some embodiments, the plasmonic sensing chip containing the functionalized sensors may be inserted into the apparatus for analyte detection. The apparatus is configured to emit a light at a series of wavelengths onto the functionalized sensors, and to collect an optical spectrum of the light transmitted through, absorbed by, or reflected from the sensors.

In some embodiments, the method further comprises comparing collected data with a baseline data of the sensors prior to the sample exposure. The baseline data for a functionalized sensor can be collected using the apparatus for analyte detection described above. In some embodiments, the baseline data can be collected prior to exposure of the sensor to the sample. In some embodiment, the baseline data is provided for a sensor functionalized with a specific biological probe. A shift in the spectral peaks after the sample exposure indicates the binding of the target analyte with the biological probe, therefore indicating the presence the target analyte in the sample. In some embodiments, the amount of the spectral peak shift may further be interpreted to provide a quantitative or semi-quantitative measurement of the concentration of a target analyte in the sample.

In some embodiments where the sensors in the array are functionalized with different biological probes, exposure of the array to the sample can result in binding of various target analytes to the corresponding sensors. Illuminating the array of sensors with a light at a series of wavelengths would allow the collection of optical spectra of each sensor be collected and compared with the baseline data. One exposure of the sensing device chip could allow detection and identification of different target analytes.

In some embodiments, the plasmon-resonance sensing device enables point-of-care (POC) detection of target analytes and POC diagnosis of disease(s)/condition(s). In some embodiments, rapid results (about 15 min or less) may be provided.

### Methods for Detecting UTI

UTI may be detected using a sensor comprising a biological probe designed to target a nucleic acid sequence derived from a UTI-causing pathogen. The method includes exposing the sensor to a sample that may contain a nucleic acid sequence derived from one or more UTI-causing pathogens, and collecting electrical, fluorescent, absorbance, transmittance, and/or extinction data from the sensor. In some embodiments, the biological probe may be a peptide nucleic acid (PNA) probe or an oligonucleotide probe.

In some embodiments, the sensor may comprise one or more biological probes. In some embodiments, each of the biological probes may be designed to bind different target nucleic acid sequences. As such, the sensor may be able to detect multiple or various target nucleic acid sequences at once. For example, the sensor can detect the presence of any of the different UTI-causing pathogens and confirm the patient's UTI diagnosis. That means UTI may be diagnosed regardless of which of the various UTI-causing pathogens is present. In some embodiments, the sensor may be able to detect and identify one or more specific pathogens that causes the UTI in a patient. This information may be useful for determining a proper or the most effective treatment option.

In some embodiments, a single biological probe can bind nucleic acids derived from more than one UTI-causing pathogens. In some embodiments, a single biological probe can bind more than one nucleic acid derived from one UTI-causing pathogen. In some embodiments, a single biological probe can bind one or more nucleic acid sequences specific to antibiotic resistance genes. In some embodiments, the biological probe may be designed to bind nucleic acid sequences from more than one antibiotic resistance genes. As a result, a sensor comprising one or more biological probes may be able to detect multiple UTI-causing pathogens. In some embodiments, a sensor comprising one or more biological probes may be able to identify one or more antibiotic resistance genes of the UTI-causing pathogens.

The biological probe may be designed or selected using computational and/or bioinformatic methods. These methods allow for rational selection of probe sequences that align upon known sequences in the scientific literature. In some embodiments, the computational approaches utilized custom python scripts, open-access sequence databases, and thermodynamic modeling tools. In some embodiments, the biological probes contain intentionally varying degrees of mismatch with the target nucleic acids. These mismatches allow for an additional degree of freedom when measuring the presence of a target nucleic acid.

In some embodiments, the biological probes described herein are independently selected from the group consisting of SEQ ID NOS: 1-32.

The sensor may have a physical property that changes upon the binding of one or more target nucleic acid sequences to the biological probes associated with the sensor. The change in the physical property can be detected by the change in electrical, fluorescent, absorbance, transmittance, and/or extinction measurement. Some non-limiting examples of sensors may include electrochemical sensors, fluorescence-based sensors, resistive sensors, and optical sensors.

### Nanoplasmonic Sensor for Pathogen/UTI Detection

A nanoplasmonic sensor for detecting urinary tract infection-causing pathogens is also described. In some embodiments, the nanoplasmonic sensor comprises an array of functionalized sensors, wherein each of the functionalized sensors in the array comprises an array of nanostructures conjugated to a biological probe/capture ligand, such as a peptide nucleic acid (PNA) probe or an oligonucleotide probe. In some embodiments, the biological probe is configured to detect the presence of a pathogen associated with urinary tract infection. In some embodiments, the pathogen is a urinary tract infection-causing pathogen. In some embodiments, the biological probe is configured to detect the presence of a urinary tract infection-causing pathogen using a specific marker associated with that given pathogen. In some embodiments, the specific marker is derived from the urinary tract infection-causing pathogens. In some embodiments, the specific marker is from a subject's response to infection by the urinary tract infection-causing pathogen.

In some embodiments, more than one functionalized sensors in the array are capable of detecting a pathogen associated with urinary tract infection in a sample. In some embodiments, at least two of the functionalized sensors in the array comprise the same biological probe for detecting a urinary tract infection-causing pathogen. In some embodiments, at least two of the functionalized sensors in the array comprise the same biological probe for detecting the same marker for a urinary tract infection-causing pathogen. In some embodiments, all of the functionalized sensors in the array comprise the same biological probe for detecting a urinary tract infection-causing pathogen.

In some embodiments, at least one of the functionalized sensors in the array comprises a different biological probe for detecting a different strains, segments, particles, mutants, and/or species of urinary tract infection-causing pathogen from the other functionalized sensors. In some embodiments, multiple sensors in the array are functionalized with different biological probes, which may allow the detection of more than one urinary tract infection-causing pathogens. In some embodiments, all of the functionalized sensors in the array comprise different biological probes from one another for detecting different urinary tract infection-causing pathogens. In some embodiments, the nanoplasmonic sensor is configured to simultaneously detect multiple strains, segments, particles, mutant, and/or species of the urinary tract infection-causing pathogens. In some embodiments, each of the functionalized sensors in the array comprises a different biological probe.

In some embodiments, a functionalized sensor may be functionalized with a negative control biological probe. The negative control biological probe may be designed to be complementary to a synthetic sequence of DNA/RNA that does not exist naturally. The negative control functionalize sensor will be expected to always return a negative result.

In some embodiments, a functionalized sensor may be functionalized with a positive control biological probe. The positive biological probe would be complementary to a synthetic sequence of DNA. A low concentration of that sequence of DNA may be spiked into the sample early in the reaction. This will indicate if the sample prep and fluid handling do enable a known concentration of target DNA to reach the sensor, indicating successful assay operation.

It will be understood that a urinary tract infection-causing, or urinary tract infection-associated pathogen can be any type of microbe capable of cultivating along the urinary tract. Non-limiting examples of pathogens include Escherichia coli, Klebsiella pneumoniae, Proteus mirabilis, Enterococcus faecalis, Staphylococcus saprophyticus, and any antibiotic-resistant strain or identified resistance gene thereof. In some embodiments, the antibiotic-resistant strain or identified resistance gene includes but is not limited to vancomycin-resistant Enterococcus faecium.

It will be understood that the biological probe can comprise any peptide and/or nucleic acid sequence or oligonucleotide sequence capable of binding to/associating with a segment of a pathogen DNA. In some embodiments, the biological probe sequence is complementary to a segment of the pathogen DNA sequence and can hybridize with the target pathogen DNA sequence when present in the sample. In some embodiments, the probe comprises one or more of a protein, peptide strand, amino acid, RNA strand, DNA strand, or and/or nucleotide. In some embodiments, the biological probe comprises one or more of a modified protein, modified peptide, modified amino acid, modified RNA strand, modified DNA strand, and/or modified nucleotide. In some embodiments, the biological probe comprises at least one of: a peptide-nucleic acid, an aptamer, an antibody, an antibody fragment, a complementary DNA, and/or an enzyme. In some embodiments, the probe is selected from the group consisting of a peptide-nucleic acid, an aptamer, an antibody, an antibody fragment, a complimentary DNA, and an enzyme. In some embodiments, the biological probe may be a PNA probe or an oligonucleotide probe having a sequence selected from the group consisting of SEQ ID NOS: 1-32. The probe sequences for target pathogens and resistance genes are listed in Table 5.

The nanostructure arrays are as disclosed herein. The nanostructures comprise a metal. In some embodiments, the nanostructures comprise a single metal. In some embodiments, the nanostructures comprise a mixture of metals. In some embodiments, the nanostructures comprise silver. In some embodiments, the nanostructures comprise copper. In some embodiments, the nanostructures comprise gold. The nanostructures in the sensors can be functionalized with the biological probes using the automatic pipetting system and method as described herein.

Also disclosed herein is a method for detecting the presence of one or more urinary tract infection-causing pathogens. The method comprises exposing the nanoplasmonic sensor of any of the embodiments disclosed herein to a sample, illuminate a light at a series of wavelengths onto each of the functionalized sensors, and collecting absorbance, transmittance or extinction data of each of the functionalized sensors. In some embodiments, the sample is a bodily fluid sample of a patient suspecting of having urinary tract infection. In some embodiments, the bodily fluid sample may be urine, blood, sweat, saliva, plasma, and/or mucus. In some embodiments, the bodily fluid sample comprises urine. In some embodiments, the light for eliminating the functionalized sensors may be emitted from a light source in an apparatus for analyte detection. The light source may be configured to emit a series of wavelengths for illuminating the sensor. For example, the series of wavelengths includes wavelengths ranging from 500-1000 nm.

In some embodiments, the method further comprises comparing the collected absorbance, transmittance, and/or extinction data of each functionalized sensor with a baseline data of each of the functionalized sensors prior to exposure to the sample. In some embodiments, the comparing step reveals an optical peak shift when an at least one urinary tract infection-causing pathogen is detected. The baseline data of the functionalized sensor includes the absorbance/transmittance measurements of functionalized sensors made prior to exposure to the sample. The peak absorbance wavelength of the functionalized sensor (prior to bonding with a target analyte) is identified. The absorbance/transmittance of the sensors are made again after exposing to the sample, and a shift in peak absorbance can be observed if a target analyte is present in the sample and binds with the probe on the functionalized sensors. The shift represents the detection signal. In some embodiments, the amount of the optical peak shift is correlated to the concentration of pathogen in the sample. In some embodiments, the amount of the optical peak shift is correlated to the concentration of the urinary tract infection-causing pathogen in the bodily fluid sample.

In some embodiments, two or more of the functionalized sensors may comprise the same biological probe. In some embodiments, at least one of the functionalized sensors may comprise different biological probes. In some embodiments, each of the functionalized sensors may comprise different biological probes. In some embodiments, when one or more of the functionalized sensors in the array comprises different biological probes, multiple strains or species of the urinary tract infection-causing pathogens can be detected simultaneously (i.e., with the same nanoplasmonic sensor/test kit). In some embodiments, the method may be performed at the point of care—that is at the location of the patient care, such as at the physician's offices, clinics, hospitals, or long-term-care facilities, patient's home, etc.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms “comprising,” “including,” “containing,” and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

The term “analyte” refers to a substance or chemical constituent that is of interest. For examples, analyte may include biological or chemical substance that may be detected by a sensing device and may be of interest for diagnosing a disease or a condition.

The term “nanostructure,” as used herein, has its standard scientific meaning and thus refers to any structure that is between about molecular size, to about microscopic size. Nanostructures comprise nanomaterials, which can be any material in which a single unit is sized at about 1 nm to about 200 nm. Nanostructures include nanoparticles, nanorods, nanosquares, nanocubes, gradient multilayer nanofilm (GML nanofilm), icosahedral twins, nanocages, magnetic nanochains, nanocomposite, nanofabrics, nanofiber, nanoflower, nanofoam, nanohole, nanomesh, nanopillar, nanopin film, nanoplatelet, nanoribbon, nanoring, nanobipyramids, irregular nanoparticles, nanosheet, nanoshell, nanotip, nanowire, and nanostructured film. It will be understood that a nanostructure can have various geometric shapes and properties based on the components of that nanostructure.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, embodiment embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The technology described herein is further illustrated by the following examples which in no way should be construed as being further limiting.

### Example 1: Electromagnetic Simulation

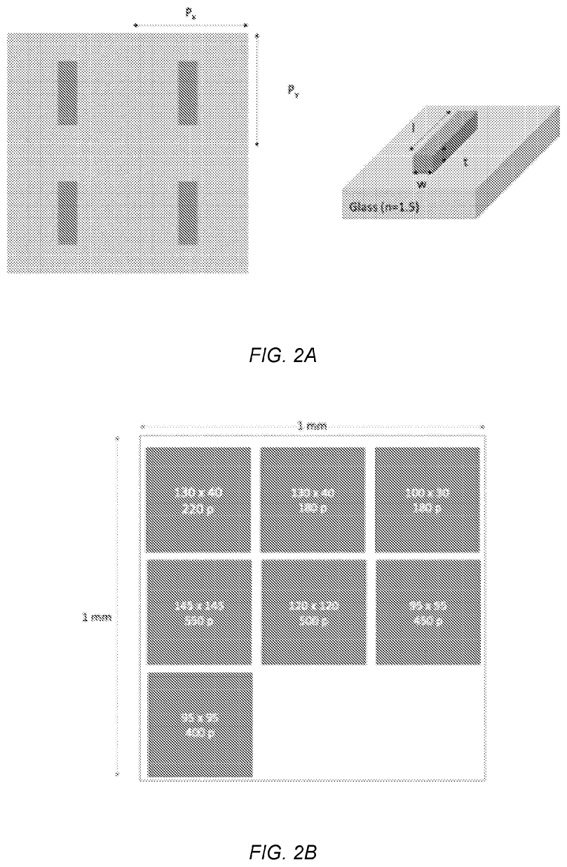

Several geometries for simulation and testing included some nanorods and some coupled nanoarrays. The nanorods is designed to reflect randomly oriented colloidal nanorods dispersed onto a glass slide. The coupled nanoarrays are designed to create surface lattice resonances. The seven geometries for a fabrication dose test are as shown in Table 1 and FIGS. 2A and 2B. FIG. 2A shows a grid with labeled dimensions for length (1), width (w), thickness (t), and spacing/periodicity (p) of the nanorods. FIG. 2B is a map of arrangement of the nanorod array within a sensor unit. As show in Table 1, the test geometries T1-T3 are nanorods and the test geometries T4-T10 are coupled nanoarrays.

TABLE 1
Test Geometries. Length, width, periodicity,
and thickness of the dose matrix test.
All dimensions are listed in nanometers.

| | Length | Width | Thickness | Spacing |
|---|---|---|---|---|
| Sample | (l), nm | (w), nm | (t), nm | (p), nm |
| T1 | 130 | 40 | 40 | 220 |
| T2 | 130 | 40 | 40 | 180 |
| T3 | 100 | 30 | 40 | 180 |
| T4 | 145 | 145 | 40 | 550 |
| T5 | 120 | 120 | 40 | 500 |
| T6 | 95 | 95 | 40 | 450 |
| T7 | 95 | 95 | 40 | 400 |
| T8 | 95 | 95 | 50 | 450 |
| T9 | 120 | 120 | 40 | 500 |
| T10 | 145 | 145 | 40 | 550 |

Full-wave electromagnetic simulations were conducted using Lumerical photonic simulation software. Periodic boundary conditions were applied in the x- and y-dimensions for each of the geometries T1-T7 as shown in FIGS. 2A-2B. For bulk sensing experiments, the refractive index of the surrounding media was changed. For PNA-DNA binding experiments, conformal shell layers of defined refractive index were modeled atop the nanostructures. Extinction and transmittance curves were returned for the wavelength range of 400-1200 nm.

### Example 2: Simulation Setup and Defining Figure-of-Merit

To study the plasmonic resonance shape as well as sensitivity to changes in refractive index, initial simulations included a bulk refractive index sensitivity analysis. In an initial iteration, gold nanorods with a wide spacing designed to represent the ordered nanoarrays was tested.

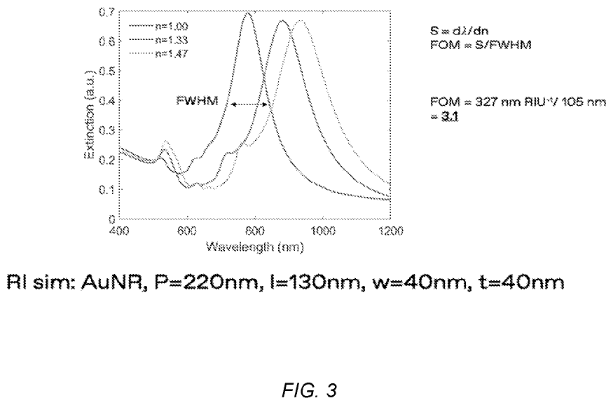

The resonances were modeled in air, water, and glycerol (increasing refractive index) and the peak locations were calculated for each of the extinction curves. This allowed for development of a sensor figure-of-merit (FOM) that considers the peak shift(s) and the narrowness of the resonances (full width at half maximum—FWHM) as shown in FIG. 3. The figure of merit was defined as the shift over full width at half maximum, allowing for a direct comparison between various geometries. A larger figure of merit represents better sensing performance due to (1) larger peak shifts for the same refractive index change, and (2) easier discrimination of peak shifts due to a narrow resonance curve. This analysis was repeated for all geometries considered.

### Example 3: PNA-DNA Binding Simulation

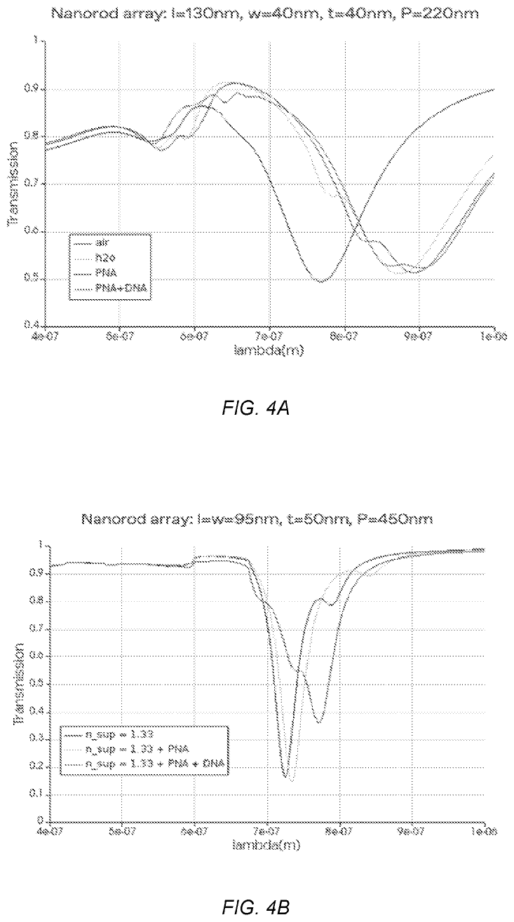

Another method of simulating these nanostructures involved simulating conformal layers with the same refractive indices expected of peptide nucleic acid (PNA) probes and PNA probes bound to DNA. We observed that the shift upon PNA+DNA binding for the surface lattice geometry (as shown in FIG. 4B) is much more apparent than the shift for the disperse nanorod geometry (as shown in FIG. 4A). These simulations point to expected shifts associated with DNA biosensing for each geometry.

### Example 4: Nanosensor Fabrication

Electron-beam lithography is a common method for patterning precise nanoscale features onto a substrate. Typically, such patterns are processed onto silicon wafers, which are optically opaque and highly conductive. For the transmittance-mode operation of the sensor, the nanostructures were configured to sit atop a transparent quartz wafer. A protocol for nanoscale patterning onto a transparent, non-conductive surface was developed.

First a thin layer of a conductive photoresist was spin-coated on the transparent quartz wafer before exposure to the pattern with an electron beam (JEOL E-beam microscope). After this, a thin (˜5 nm) chromium adhesion layer was thermally evaporated onto the patterned substrate, followed by a thicker (about 40-50 nm) pure gold layer. Chemical liftoff was conducted to form the nanostructures array before dicing the substrate for testing. The first sample produced with this pattern was a dose matrix test to evaluate the power of the electron beam. After this parameter was identified, all future processes were conducted under the same conditions.

### Example 5: Simulation of Selected Geometries

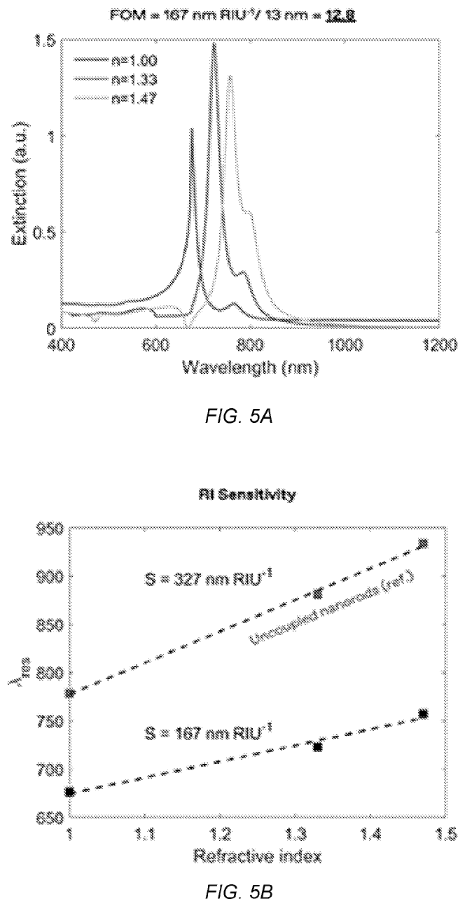

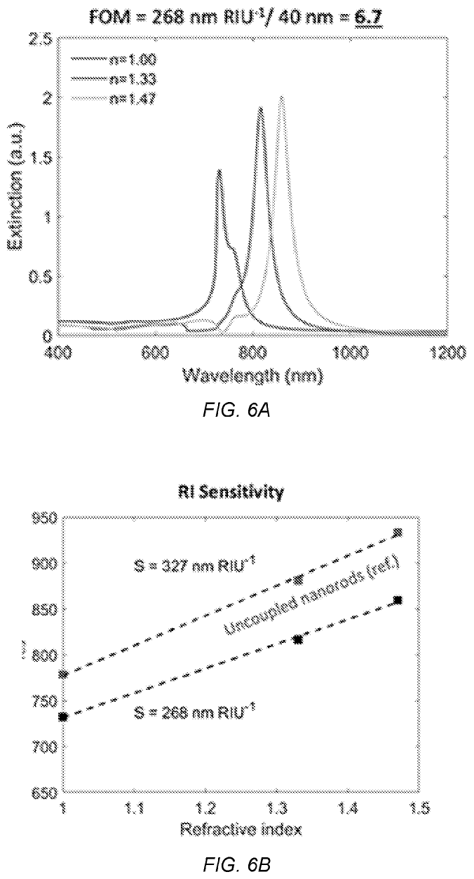

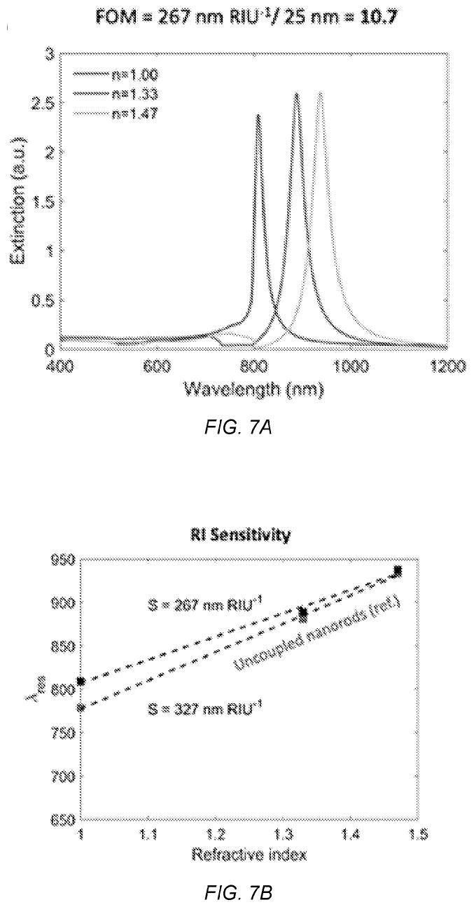

Bulk refractive simulations were conducted on sample geometries T8-T10 described in Table 1. Transmittance through the samples was measured using an optical readout instrumentation. Wavelength bounds were set from 450 nm to 950 nm. For seamless integration with the readout instrumentation, the individual sensor of the sensing device was fabricated to have a 1 mm² area of nanostructures array to fully align with the light source spot size and minimizing signal loss. The results for the three surface lattice resonance geometries (T8-T10) are as shown in FIGS. 5A/B, 6A/B, and 7A/B, respectively. Both the shape of the peak and the refractive index peak shifts are shown. The calculated figure-of-merits for T8-T10 were 12.8, 6.7, and 10.7, respectively. Further, the refractive index sensitivities of each of these geometries are shown in FIGS. 5B, 6B, and 7B. All sensitivities are compared to the 140 nm×40 nm 220 p sample labeled “uncoupled nanorods”. A higher slope indicates better sensing performance. Sample geometry T10 is the highest performance due to its high figure of merit (10.7) and its relatively high refractive index sensitivity (267 nm/RIU).

### Example 6: Comparison of Simulation and Experiment

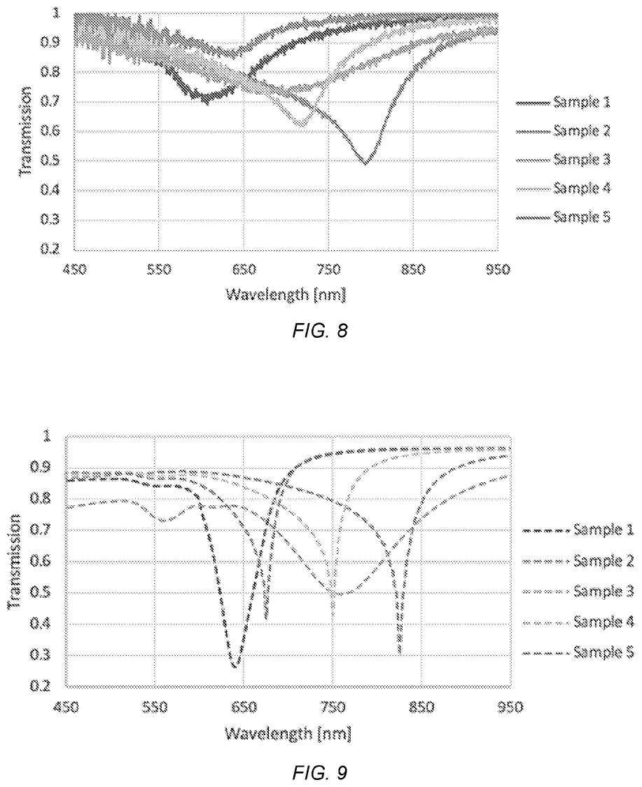

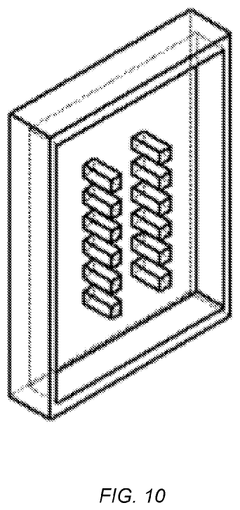

Nanostructure array samples 1-5 were fabricated with the nanostructure dimensions shown in Table 2. The transmittance of each sample was experimentally measured (shown in FIG. 8) and compared to the peak shape from the simulations (shown in FIG. 9). There was found to be exceptional agreement between the experimental and simulation data, including the peak shape and resonance location.

TABLE 3
Dimensions of nanostructure arrays.

| | Length | Width | Thickness | Spacing |
|---|---|---|---|---|
| Sample | (l), nm | (w), nm | (t), nm | (p), nm |
| 1 | 96 | 96 | 50 | 400 |
| 2 | 145 | 145 | 50 | 550 |
| 3 | 130 | 130 | 50 | 220 |
| 4 | 120 | 120 | 50 | 500 |
| 5 | 95 | 95 | 50 | 450 |

The present disclosure also puts forth a methodology for rational design of regularly spaced nanoparticle arrays for plasmonic sensing. The Applicants tested 5-7 geometries through both simulation and experimental analysis, and finally selected the 145 nm×145 nm Through both simulation and experimental analysis, a nanoarray geometry that shows high-amplitude resonance and refractive index sensitivity may be selected for the production of the plasmonic-resonance sensing device.

### Example 7: Functionalization of Nanostructures

A 2×6 array of 1 mm² sensors (12 sensors total) was functionalized with peptide-nucleic acid (PNA) probes. Each of the sensors contains an array of 145 nm×145 nm gold nanostructures with regular spacing. In order to individually functionalize the sensor arrays to be target-specific, a polydimethylsiloxane (PDMS) polymer micro-well array was fabricated. This micro-well array was aligned with the substrate such that each sensor could be accessed through a single micro-well. This approach created repeatable, programmable coordinates for the automatic pipetting system (e.g., Integra ASSIST PLUS pipetting robot).

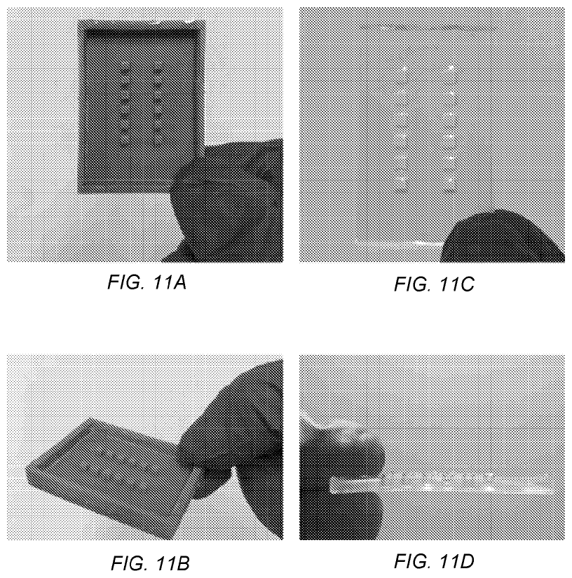

The micro-well structure atop the sensing array allowed for individual fluid delivery to each sensing spot, enabling multiplexing of up to 12 targets on a single sensing chip. To this end, a mold was designed using Solidwaorks CAD to allow for fabrication of a polymer micro-well array that align with the coordinates of the sensors (FIG. 10). The mold for casting the PDMS micro-wells was designed in Solidworks consisting of twelve 2 mm×2 mm×5 mm (20 mm³) pillars. The pillars were positioned to match the coordinates of sensor array on the glass substrate. Master molds, as shown in FIGS. 11A and 11B, were then made using SLA 3D printing.

Micro-well array devices were fabricated in the molds using PDMS soft lithography. Sylgard 184 silicone elastomer, base and curing agent (Dow Corning, Midland, MI) were mixed in a ratio of 10:1, by weight. Next, the PDMS prepolymer was cast on the master mold and cured at 80° C. in a convection oven for approximately 1.5 h. The cured PDMS micro-well array, as shown in FIGS. 11C and 11D, was removed from the master mold. The polymer micro-well array was affixed atop the sensor array using washable glue, enabling removable bonding for sensor reuse. This entire system was attached to a standard 75×25 mm microfluidic chip and was then ready for molecular detection.

### Example 8: Automated Robotic Functionalization of Sensors

The prepared plasmonic sensing chip was integrated with the automatic pipetting system (e.g., Integra ASSIST Plus) for surface functionalization. To covalently functionalize the sensor with selected biological probes, such as PNA probes, the gold nanostructures on a glass substrate were first incubated with 1 mg/mL dithiobis succinimidyl propionate (DSP) dissolved in dimethyl sulfoxide (DMSO) for 20 min. This crosslinking molecule activated the gold surface to enable coupling of free amines on the PNA. Next, the sensor arrays were put in contact with 1 mg/mL PNA probe dispersed in Tris-EDTA buffer (pH 7.0) for 30-45 min. Transmission spectra were collected before and after conjugation to characterize successful PNA conjugation.

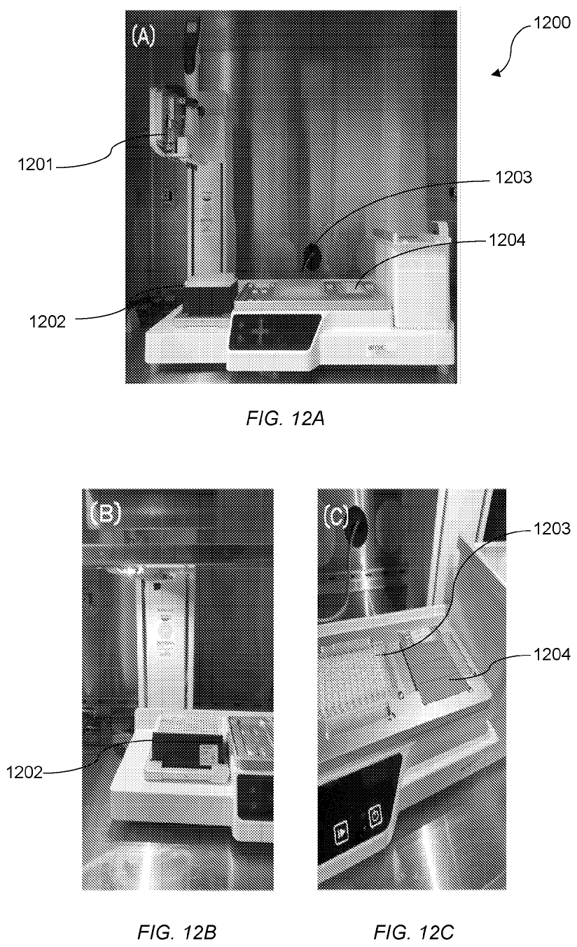

The sensor functionalization process described above was automated using an Integra ASSIST PLUS pipetting robot. In order to effectively position the device onto the deck of the Integra ASSIST PLUS pipetting robot, we designed and fabricated (3D printed) a custom 4-slot microscope slide holder/adaptor the size of a standard 96-well plate. This adapter could readily be integrated with the liquid handler's robotic deck. A 96-well plate was pre-loaded with functionalization reagents and placed in the robot's aspiration deck. To start up the machine, a Voyager electronic 125 μL, 8-channel pipette was loaded onto the robot. A suite of six programs were developed to aspirate, dispense, and clear tips in an automated fashion. These custom programs allow for multiplexed functionalization of twelve PNAs upon the sensor arrays. Table 4 shows 6 programs for automated functionalization of the sensors using the pipetting robot. The programs indicate pipette tip location, 96-well plate location, aspiration volume, and dispense volume for each stage. FIG. 12A is a photo of the Integra ASSIST PLUS pipetting robot 1200, with pipette holder 1201 on the left, tip box 1202, 96-well plate holder 1203, and custom chip adapter 1204. FIG. 12B depicts the tip box 1202 aligned under pipette holder 1201. FIG. 12C depicts the 96 well plate 1203 and adapter 1204 during functionalization.

TABLE 4
Integra ASSIST custom programs.

| | | | | | Dispense |
|---|---|---|---|---|---|
| | | Pipette | 96 Well | Application | Volume |
| | Program | Tip | Palate | Volume | (μL)/ |
| # | Name | Location | Location | (μL) | Sensor Well |
| 1 | Dispense TE | A1, B1 | A1, B1 | 80 | 12 |
| 2 | Aspirate TE | I1, J1 | X | 12 | X |
| 3 | Dispense DSP | A2, B2 | A2, B2 | 80 | 12 |
| 4 | Aspirate DSP | I2, J2 | X | 12 | X |
| 5 | Dispense PNA | A, B(3-5); | A(3-8), | 15 | 12 |
| | | I, J(3-5) | B(3-8) | | |
| 6 | Aspirate PNA | A, B(6-8); | X | 12 | X |
| | | I, J(6-8) | | | |

First, the Tris-EDTA (TE) buffer is dispensed and removed from the chip surface to clean the surface and to ensure a tight seal of the micro-well array onto the sensing substrate. Then DSP, a bivalent cross-linking molecule, is introduced to the chip surface and readily adsorbs to the gold surface within 15-20 minutes. The presence of active NHS groups enables cross-linking to proteins (i.e., PNAs). Examples of linkers for attaching a capturing ligand/biological probe (such as PNA) are presented in Table 5. Finally, the DSP is aspirated and the PNA probes are directly dispensed atop the sensing surface and couple to the free amines on the nanostructures. After the excess PNA solution is aspirated, the chip is covalently functionalized with PNAs and ready to use for sample testing.

TABLE 5
Attachment of Ligand to Gold.

| Active Group | Cross-linking molecules |
|---|---|
| on Ligand | for conjugation to gold |
| Amine | DSP (dithiobis(succinimidyl propionate)) |
| Cysteine | Direct attachment of thiol group |
| Thiol | Direct attachment of thiol group |
| Amine | EDC (1-ethyl-3-(3-dimethylaminopropyl)carbodiimide |
| | hydrochloride) and NHS (N-hydroxysulfosuccinimide) |
| Amine | p-mercaptobenzoic acid (p-MBA) self-assembled |
| | monolayer followed by EDC and NHS |

While pipetting robot systems have been widely utilized for fluid loading applications, to the best of our knowledge this is the first time such a system has been employed for covalently attaching a molecular capture probe to a solid-state sensor. We accomplish this through successive dispense and aspiration steps atop the sensors.

### Example 9: Development of UTI probes

In silico methods were used in designing PNA probes for nanoplasmonic characterization of UTI causing pathogens. Six PNA probes specific to the pathogenic genomes of Escherichia coli, Klebsiella pneumoniae, Enterococcus spp, Escherichia coli carrying Extended-spectrum beta lactamases (ESBL), and Vancomycin resistant Enterococci (VRE) were designed. Two probes for VRE detection were designed in which one is exclusive for vanA gene and the other is consensus to both vanA and vanB genes. ESBL-Ecoli probe was designed to detect the gene group belonging to bla_{CTX-M-1} that is inclusive of 113 CTX-M alleles as determined by the described in silico analysis.

Genes that are conserved within the desired group of targeted pathogens, but distinct enough from their nearest neighbors were determined through literature survey and visual examination of the alignments for species identification. Reference sequences for AMR genes were retrieved from NCBI database.

Reference target genes were subjected to BLAST against 5000 records in the nucleotide database to generate XML files containing complete results of alignments of homologous sequences (coverage/identity >80%). The XML files containing the alignment records were parsed into python using Biopython modules. Identical sequence records were grouped indicating the number of repeats and parsed into fasta files. Fasta files were used to realign the sequence records for further analysis.

The sequence alignments were visually examined to identify potential locations for probe placement. PNA probes were designed such that the Tₘ of the PNA-DNA duplex is ˜80 C. The probe lengths were kept <25 nt. The Tm of the PNA-DNA hybrid was determined as previously described¹ using the PNA Bio Tool. Purine composition was kept <50% to avoid precipitation of PNA probes. Sequences that produce stable homodimers, and hairpins (Tm>30° C.) were avoided.

Once the probe sequence was determined, the analytical inclusivity of the given probe was evaluated using multiple databases. All probes were tested against the NCBI's nucleotide database to retrieve a complete record of high-scoring pairs (HSPs). Parameters including Accession Number, Identity, Coverage, Number of mismatches, mismatched based and location, was retrieved using custom scripts. Identical results were grouped marking a single representative record and the number of records that duplicates the parameters. Additionally, based on the target, additional databases were used to further validate inclusivity/cross-reactivity using the same analysis criteria. Accordingly, all probes targeting UTI-causing pathogens were tested against the NCBIs prokaryotic representative genome database. Additionally, probes that target AMR genes were tested against the BioProject 313047 sequence records that contains curated representative genomes that carry AMR genes.

Target genes for E. coli, K. pneumoniae and Enterococcus spp were selected based on previously reported analyses. Summary of the UTI probes is as shown in Table 5.

TABLE 5
Summary of UTI Probes

| SEQ ID | Probe Name | Sequence | Target |
|---|---|---|---|
| 1 | Enterococcus | ATACATGCAAGTCGAACGCTTCTTT | 16S rRNA |
| 2 | Escherichia coli | CCACAAACCGTTCTACTTTACTGGC | uidA |
| 3 | Klebsiella pneumoniae | TCACCCTGTTTGATGGCCGCACC | rpoB |
| 4 | Vancomycin-resistant | CTGAGCTTTGAATATCGCAGCCT | vanA |
| | Enterococci-vanA | | |
| 5 | Vancomycin-resistant | CCGACCTCACAGCCCGAAA | vanA/vanB |
| | Enterococci-vanA/B | | |
| 6 | ESBL-producing | CGGTTCGCTTTCACTTTTCTTCAGC | blaCTX-M-1 |
| | Escherichia coli | | |
| 7 | VRE_van | ACTGAGCTTTGAATATCGCAGCCT | vanA |
| 8 | ESBL_CTXM1 | CGGTTCGCTTTCACTTTTCTCAGC | CTX-M-1 |
| 9 | Enterococcus_16s | ATACATGCAAGTCGAACGCTTCTTT | E. faecalis and E. |
| | | | faccium |
| 10 | Ecoli_uidA | CCACAAACCGTTCTACTTTACTGGC | E. coli |
| 11 | Kp_rpoB | TCACCCTGTTTGATGGCCGCACC | K. pneumoniae |
| 12 | Vre_VanAB | CCGACCTCACAGCCCGAAA | vanAB |
| 13 | Ssap_agr | AATGCGAACCAAATATGCC | S. saprophyticus |
| 14 | Saga_GroEL | CAAGTTTTAGGACAGTCTGCT | S. agalactiae |
| 15 | Pmir_ureR | GGGATCGCATTAAATTACCT | P. mirabilis |
| 16 | S. aureus PNA1 | GCGATTGATGGTGATACGGTT | S. aureus |
| 17 | P. aeruginosa | GGGGGATCTTCGGACCTCA | P. aeruginosa |
| 18 | E. coli PNA2 | TGGTAATTACCGACGAAAACGGC | E. coli |
| 19 | N. gonorrhoeae porA | CCGGAACTGGTTTCATCTGATT | N. gonorrhoeae |
| 20 | 16S Consensus | CTGAGCCAGGATCAAACTCT | broad spectrum |
| 21 | Kp20 | TCACCCTGTTTGATGGCCGC | K. pneumoniae |
| 22 | Kp15 | TCACCCTGTTTGATG | K. pneumoniae |
| 23 | Kp10 | TCACCCTGTT | K. pneumoniae |
| 24 | NG_16s_1 | TTGCCAATATCGGCGGCC | N. gonorrhoeae |
| 25 | NG_16s_2 | CCAATATCGGCGGCC | N. gonorrhoeae |
| 26 | NG-PorA_2 | AACTGGTTTCATCTG | N. gonorrhoeae |
| 27 | NG_gyrA_WT_1 | TCCGCAGTTTACGAC | N. gonorrhoeae |
| | | | gyrA |
| 28 | NG_gyrA_MUT_1 | TTCGCAGTTTACGGC | N. gonorrhoeae |
| | | | gyrA |
| 29 | EC_16S | TCAATGAGCAAAGGTC | E. coli |
| 30 | SA_16S | GCCCTTTGTATTGTCCAC | S. aureus |
| 31 | KP_16S | CACCTACACACCAGCC | K. pneumoniae |
| 32 | PSA_16S | AACTTGCTGAACCAC | P. aeruginosa |

Two probes were designed targeting Vancomycin-resistant Enterococci in which one probe exclusively targets the vanA gene while the other binds to both vanA and vanB genes. The probe for ESBL producing E. coli targets the gene markers belonging to the bla_{CTX-M-1} group. The analytical inclusivity and cross-reactivity of the probes were evaluated against the nucleotide (Table 6), representative prokaryotic genomes (Table 7) and against the Bioproject 313047 database for AMR targets (Table 8). The full list of allele inclusivity for ESBL-Ecoli is shown in Table 9. This data demonstrates all alleles that can be bound by the designed ESBL-Ecoli probe shown in Table 5.

TABLE 6
Inclusivity/Cross-reactivity as determined with the nucleotide database. The results are
sorted based on the number of counts and only the first 6 records per probe are shown.

| | | | | | | |
|---|---|---|---|---|---|---|
| Species | Accession | mismatches | Identity | Coverage | Strand | count |

E. coli (uidA)

| | | | | | | | |
|---|---|---|---|---|---|---|---|
| 0 | Escherichia coli | CP054236.1 | 0 | 100% | 100% | 1 | 2774 |
| 1 | Escherichia coli | CP054353.1 | 1 | 96% | 100% | 1 | 101 |
| 2 | Escherichia coli | CP054224.1 | 4 | 84% | 84% | 1 | 100 |
| 3 | Shigella flexneri | CP055124.1 | 0 | 100% | 100% | 1 | 86 |
| 4 | Escherichia sp. | KR424443.1 | 0 | 100% | 100% | 1 | 77 |
| 5 | Cloning vector | MN400674.1 | 0 | 100% | 100% | 1 | 61 |

Enterococcus (16s)

| | | | | | | | |
|---|---|---|---|---|---|---|---|
| 0 | Enterococcus faecalis | CP046108.1 | 0 | 100% | 100% | −1 | 1373 |
| 1 | Enterococcus faecium | CP041261.3 | 0 | 100% | 100% | −1 | 773 |
| 2 | Enterococcus sp. | FP929058.1 | 0 | 100% | 100% | −1 | 493 |
| 3 | Enterococcus faecium | CP053704.1 | 1 | 96% | 100% | −1 | 387 |
| 4 | Weissella confusa | CP049097.1 | 4 | 84% | 84% | −1 | 197 |
| 5 | Enterococcus hirae | CP055232.1 | 0 | 100% | 100% | −1 | 177 |

Klebsiella pneumoniae (rpoB)

| | | | | | | | |
|---|---|---|---|---|---|---|---|
| 0 | Klebsiella pneumoniae | CP054780.1 | 0 | 100% | 100% | −1 | 1281 |
| 1 | Klebsiella variicola | CP054254.1 | 1 | 96% | 100% | −1 | 67 |
| 2 | Bacillus licheniformis | CP045814.1 | 6 | 74% | 74% | −1 | 37 |
| 3 | Phaeobacter inhibens | CP031952.1 | 4 | 83% | 83% | −1 | 22 |
| 4 | Bacillus paralicheniformis | CP043501.1 | 6 | 74% | 74% | −1 | 17 |
| 5 | Zea mays | NM_001154872.2 | 6 | 74% | 74% | 1 | 17 |

ESBL- E. coli (ctx-m-1)

| | | | | | | | |
|---|---|---|---|---|---|---|---|
| 0 | Escherichia coli | CP055257.1 | 0 | 100% | 100% | 1 | 1932 |
| 1 | Klebsiella pneumoniae | LC556222.1 | 0 | 100% | 100% | 1 | 1020 |
| 2 | Uncultured bacterium | MT215960.1 | 0 | 100% | 100% | −1 | 175 |
| 3 | Salmonella enterica | MN619286.1 | 0 | 100% | 100% | 1 | 123 |
| 4 | Synthetic construct | HQ734710.1 | 0 | 100% | 100% | −1 | 65 |
| 5 | Enterobacter cloacae | LC556220.1 | 0 | 100% | 100% | 1 | 49 |

VRE (vanA)

| | | | | | | | |
|---|---|---|---|---|---|---|---|
| 0 | Enterococcus faecium | MN478489.1 | 0 | 100% | 100% | −1 | 222 |
| 1 | Enterococcus faecium | LR135359.1 | 4 | 83% | 87% | −1 | 81 |
| 2 | Staphylococcus aureus | MN295031.1 | 0 | 100% | 100% | −1 | 40 |
| 3 | Enterococcus faecalis | MK959500.1 | 0 | 100% | 100% | −1 | 39 |
| 4 | Rathayibacter toxicus | MH633474.1 | 4 | 83% | 87% | 1 | 25 |
| 5 | Bacillus altitudinis | CP038517.1 | 6 | 74% | 74% | −1 | 20 |

VRE (vanA/vanB)

| | | | | | | | |
|---|---|---|---|---|---|---|---|
| 0 | Enterococcus faecium | MN478489.1 | 0 | 100% | 100% | −1 | 322 |
| 1 | Enterococcus faecalis | MK959500.1 | 0 | 100% | 100% | −1 | 66 |
| 2 | Staphylococcus aureus | MK095504.1 | 0 | 100% | 100% | −1 | 57 |
| 3 | PREDICTED: Corvus | XM_032095935.1 | 3 | 84% | 84% | −1 | 19 |
| 4 | PREDICTED: Rousettus | XM_016144335.2 | 3 | 84% | 84% | −1 | 18 |
| 5 | Eilema sororculum | OU618561.1 | 3 | 84% | 84% | 1 | 15 |

TABLE 7
Inclusivity/Cross-reactivity as determined with the representative
genomes for prokaryotes. Only selected results are shown here.

| | | | | |
|---|---|---|---|---|
| Species | Accession | % Identity | Mismatches | hits/record |

E. coli (uidA)

| | | | | | |
|---|---|---|---|---|---|
| 0 | Clostridium acidisoli | NZ_FWXH01000013 | 100% | 0 | 1 |
| 1 | Escherichia coli | NC_000913 | 100% | 0 | 1 |
| 2 | Escherichia coli | NC_002695 | 100% | 0 | 1 |
| 6 | Shigella boydii | NZ_LPTR01000022 | 100% | 0 | 1 |
| 7 | Shigella flexneri | NC_004337 | 100% | 0 | 1 |
| 8 | Shigella sonnei | NZ_CP055292 | 100% | 0 | 1 |

Enterococcus (16S)

| | | | | | |
|---|---|---|---|---|---|
| 39 | Enterococcus faecalis | NZ_KB944666 | 100% | 0 | 4 |
| 40 | Enterococcus faecium | NZ_CP038996 | 100% | 0 | 6 |
| 44 | Enterococcus gallinarum | NZ_KZ846567 | 96% | 1 | 4 |
| 45 | Enterococcus gilvus | NZ_CP030932 | 96% | 1 | 6 |
| 47 | Enterococcus haemoperoxidus | NZ_KB946316 | 100% | 0 | 3 |
| 50 | Enterococcus hirae | NZ_CP015516 | 100% | 0 | 5 |

Klebsiella pneumoniae (rpoB)

| | | | | | |
|---|---|---|---|---|---|
| 8 | Franconibacter helveticus | NZ_QISR01000014 | 96% | 1 | 1 |
| 9 | Klebsiella pneumoniae | NC_016845 | 100% | 0 | 1 |
| 10 | Klebsiella quasivariicola | NZ_CAAHGB010000003 | 96% | 1 | 1 |
| 11 | Klebsiella variicola | NZ_CP054254 | 96% | 1 | 1 |
| 12 | Mixta theicola | NZ_NWUO01000024 | 95% | 1 | 1 |
| 13 | Myxococcus | NZ_VIFM01000572 | 100% | 0 | 1 |

VRE (vanA)

| | | | | | |
|---|---|---|---|---|---|
| 0 | Alicyclobacillus shizuokensis | NZ_BCQV01000042 | 100% | 0 | 1 |
| 1 | Cohnella zeiphila | NZ_JACJVO010000017 | 100% | 0 | 1 |
| 2 | Enterococcus saigonensis | NZ_AP022823 | 100% | 0 | 1 |
| 3 | Halobacillus alkaliphilus | NZ_FOOG01000006 | 100% | 0 | 1 |
| 4 | Neobacillus jeddahensis | NZ_CCAS010000079 | 100% | 0 | 1 |
| 5 | Paenibacillus jilunlii | NZ_CP048429 | 100% | 0 | 1 |

VRE (vanA/vanB)

| | | | | | |
|---|---|---|---|---|---|
| 0 | Bat mastadenovirus | NC_029898 | 100% | 0 | 1 |
| 1 | Mycobacterium phage | NC_054727 | 94% | 1 | 1 |

TABLE 8
Inclusivity/Cross-reactivity against Antimicrobial Resistance
database. Only selected results are shown in here.

| | | | | | |
|---|---|---|---|---|---|
| Species | Allele | Genbank ID | % Identity | Mismatches | % Coverage |

ESBL-E. coli (CTX-M-1)

| | | | | | | |
|---|---|---|---|---|---|---|
| 0 | Escherichia coli | CTX-M-1 | NG_048897 | 100% | 0 | 100% |
| 1 | Klebsiella pneumoniae | CTX-M-10 | NG_048898 | 100% | 0 | 100% |
| 2 | Escherichia coli | CTX-M-100 | NG_048899 | 60% | 10 | 68% |
| 3 | Escherichia coli | CTX-M-101 | NG_048900 | 100% | 0 | 100% |
| 4 | Escherichia coli | CTX-M-103 | NG_048902 | 100% | 0 | 100% |

VRE (vanA)

| | | | | | | |
|---|---|---|---|---|---|---|
| 0 | Bacillus circulans | vanA | NG_048324 | 100% | 0 | 100% |
| 1 | Enterococcus faecium | vanA | NG_048323 | 100% | 0 | 100% |
| 2 | Enterococcus faecium | vanA | NG_048325 | 100% | 0 | 100% |
| 3 | Enterococcus faecalis | vanB | NG_048331 | 91% | 2 | 91% |
| 4 | Paenibacillus thiaminolyticus | vanA-Pt | NG_048327 | 87% | 3 | 87% |

VRE (VanA/VanB)

| | | | | | | |
|---|---|---|---|---|---|---|
| 0 | Enterococcus faecium | VanA | NG_048323 | 100% | 0 | 100% |
| 1 | Enterococcus faecium | VanA | NG_048325 | 100% | 0 | 100% |
| 2 | Enterococcus faecalis | VanB | NG_048331 | 100% | 0 | 100% |
| 3 | Enterococcus faecalis | VanB | NG_048332 | 100% | 0 | 100% |
| 4 | Enterococcus faecium | VanB | NG_048333 | 100% | 0 | 100% |

TABLE 9
Inclusivity of CTX-M alleles with ESBL-E. coli probe.

CTX-M-1
CTX-M-3
CTX-M-10
CTX-M-12
CTX-M-15
CTX-M-22
CTX-M-23
CTX-M-28
CTX-M-29
CTX-M-30
CTX-M-32
CTX-M-33
CTX-M-34
CTX-M-36
CTX-M-37
CTX-M-42
CTX-M-52
CTX-M-53
CTX-M-54
CTX-M-55
CTX-M-58
CTX-M-60
CTX-M-61
CTX-M-62
CTX-M-66
CTX-M-68
CTX-M-69
CTX-M-71
CTX-M-72
CTX-M-79
CTX-M-80
CTX-M-82
CTX-M-88
CTX-M-96
CTX-M-101
CTX-M-103
CTX-M-114
CTX-M-116
CTX-M-117
CTX-M-123
CTX-M-127
CTX-M-132
CTX-M-136
CTX-M-138
CTX-M-139
CTX-M-142
CTX-M-143
CTX-M-144
CTX-M-146
CTX-M-150
CTX-M-153
CTX-M-154
CTX-M-155
CTX-M-156
CTX-M-157
CTX-M-158
CTX-M-162
CTX-M-163
CTX-M-164
CTX-M-166
CTX-M-167
CTX-M-169
CTX-M-170
CTX-M-172
CTX-M-173
CTX-M-175
CTX-M-176
CTX-M-177
CTX-M-178
CTX-M-179
CTX-M-180
CTX-M-181
CTX-M-182
CTX-M-183
CTX-M-184
CTX-M-186
CTX-M-187
CTX-M-188
CTX-M-189
CTX-M-190
CTX-M-193
CTX-M-194
CTX-M-197
CTX-M-202
CTX-M-203
CTX-M-204
CTX-M-206
CTX-M-207
CTX-M-208
CTX-M-209
CTX-M-210
CTX-M-211
CTX-M-212
CTX-M-216
CTX-M-218
CTX-M-220
CTX-M-222
CTX-M-224
CTX-M-225
CTX-M-226
CTX-M-227
CTX-M-228
CTX-M-230
CTX-M-231
CTX-M-232
CTX-M-234
CTX-M-236
CTX-M-237
CTX-M-238
CTX-M-244
CTX-M-245
CTX-M-246
CTX-M-251

### Example 10: Methodology/Sample Preparation for UTI Screening

Upstream sample processing is limited to a ten-minute thermal lysis step followed by direct transfer to sensing substrate. Additionally, successful molecular sensing of target material is demonstrated in a range of sample matrices including synthetic urine and healthy human urine.

Quantitative Genomic DNA (>1×10⁵ copies/μL) was purchased from American Type Culture Collection (ATTC, Manassas, VA) for the following organisms: Extended-Spectrum Beta-Lactamase (ESBL)-producing Escherichia coli #BAA-2326 and Vancomycin-resistant Enterococcus faecium (VRE) #700221. The following bacterial strains were also purchased from ATCC and revived according to the specified protocol: Escherichia coli #25922, Klebsiella pneumoniae #13883, and Enterococcus faecalis #29212. Bacteria were cultured aerobically in 5 mL Tryptic Soy Broth (TSB) (Becton Dickinson, Franklin Lakes, NJ) (E. coli, K. pneumoniae) or 5 mL Brain Heart Infusion (BHI) (Becton Dickinson, Franklin Lakes, NJ) (E. faecalis) in a 50 mL conical tube for ˜8 hours (37° C., 250 rpm, shaking). Cultures were centrifuged (12,100×g, 4° C. 10 min) and the supernatant was aspirated. Cultures were resuspended in fresh phosphate buffered saline (PBS). Next, 100 μL of bacterial suspension was added to a 1.5 mL microcentrifuge tube containing 900 μL of desired sample matrix.

Bacterial samples were quantified using traditional 10 μL drop plates (Table 10).

TABLE 10
Plate counts for data, as quantified using 10 μL drop plates (n = 3).

| | Average |
|---|---|
| Species | CFU/mL |
| E. coli #1 in PBS & Synthetic Urine | 1.62E9 |
| E. coli #2 in PBS | 1.35E9 |
| E. coli #3 in PBS | 1.15E9 |
| K. pneumoniae #1 in PBS & Synthetic Urine | 8.97E8 |
| K. pneumoniae #2 in PBS | 1.06E9 |
| K. pneumoniae #3 in PBS | 1.03E9 |
| E. faecalis #1 in PBS & Synthetic Urine | 2.57E8 |
| E. faecalis #2 in PBS | 1.03E8 |
| E. faecalis #3 in PBS | 1.20E8 |
| E. coli @ ~102 in Synthetic Urine | 1.62E2 |
| E. coli @ ~103 in Synthetic Urine | 1.62E3 |
| E. coli @ ~104 in Synthetic Urine | 1.62E4 |
| E. coli @ ~105 in Synthetic Urine | 1.62E5 |
| E. coli @ ~106 in Synthetic Urine | 1.62E6 |
| E. coli @ ~107 in Synthetic Urine | 1.62E7 |
| K. pneumoniae @ ~102 in Synthetic Urine | 8.97E1 |
| K. pneumoniae @ ~103 in Synthetic Urine | 8.97E2 |
| K. pneumoniae @ ~104 in Synthetic Urine | 8.97E3 |
| K. pneumoniae @ ~105 in Synthetic Urine | 8.97E4 |
| K. pneumoniae @ ~106 in Synthetic Urine | 8.97E5 |
| K. pneumoniae @ ~107 in Synthetic Urine | 8.97E6 |
| E. faecalis @ ~102 in Synthetic Urine | 2.57E2 |
| E. faecalis @ ~103 in Synthetic Urine | 2.57E3 |
| E. faecalis @ ~104 in Synthetic Urine | 2.57E4 |
| E. faecalis @ ~105 in Synthetic Urine | 2.57E5 |
| E. faecalis @ ~106 in Synthetic Urine | 2.57E6 |
| E. faecalis @ ~107 in Synthetic Urine | 2.57E7 |
| E. coli @ ~104 in Human Urine | 2.33E4 |
| E. coli @ ~105 in Human Urine | 2.33E5 |
| E. coli @ ~106 in Human Urine | 2.33E6 |
| K. pneumoniae @ ~104 in Human Urine | 1.33E4 |
| K. pneumoniae @ ~105 in Human Urine | 1.33E5 |
| K. pneumoniae @ ~106 in Human Urine | 1.33E6 |
| E. faecalis @ ~104 in Human Urine | 2.33E4 |
| E. faecalis @ ~105 in Human Urine | 2.33E5 |
| E. faecalis @ ~106 in Human Urine | 2.33E6 |

Synthetic urine solution was purchased from Fisher Scientific (Hampton, NH) and manufactured by Ricca Chemical (Arlington, TX), and autoclaved prior to use. Healthy patient urine was obtained from five volunteers. Urine samples were transported in a cooler and refrigerated for <˜2 hours prior to suspending bacteria or quantitative genomic DNA. Additionally, participant urine samples were plated out on Tryptic Soy Agar (TSA) plates to roughly quantify any bacteria present in the sample prior to spiking in target organism (Table 11).

TABLE 11
Plate counts for participant urine sample, as quantified
using 500 μL spread plates (n = 1)

| Collection | Participant | Approximate bacterial |
|---|---|---|
| Day | # | load (CFU/mL) |
| Day 1 | 1 | ~1000 |
| Day 1 | 2 | ~340 |
| Day 1 | 3 | ~70 |
| Day 1 | 4 | ~2000 |
| Day 1 | 5 | ~70 |
| Day 2 | 1 | ~30 |
| Day 2 | 2 | ~1000 |
| Day 2 | 3 | ~3000 |
| Day 2 | 4 | ~1000 |
| Day 2 | 5 | ~1000 |
| Day 3 | 1 | ~30 |
| Day 3 | 2 | ~1000 |
| Day 3 | 3 | ~2000 |
| Day 3 | 4 | ~20 |
| Day 3 | 5 | ~50 |

Fresh urine samples were collected on three days with experiments distributed as follows: (Day 1: E. coli #25922; Day 2: E. faecalis #29212 and Vancomycin-resistant Enterococcus (VRE) #700221; Day 3: K. pneumoniae & Extended-Spectrum Beta-Lactamase (ESBL)-producing E. coli #BAA-2326). All urine samples (15 total) have been stored long-term at −20° C.

For functionalization, the gold nanostructures on a glass slide were incubated with 1 mg/mL dithiobis succinimidyl propionate (DSP) dissolved in dimethyl sulfoxide (DMSO) for 30 minutes. This crosslinking molecule activated the gold surface for coupling of free amines on the PNA. Then, the sensor arrays were put in contact with 1 mg/mL PNA probe dispersed in Tris-EDTA buffer (pH 7.0) for 30 minutes. Transmission spectra were collected before and after conjugation to quantify successful PNA conjugation. The nanosensor functionalization process was automated using an Integra ASSIST PLUS pipetting robot.

For bacterial samples, 1.5 mL microcentrifuge tubes containing 1 mL fluid volumes were placed into a heating block for 10 minutes at 100° C. Samples cooled at room temperature for approximately 5 min. Bacterial lysate was diluted to desired concentration in desired sample matrix. Next, 8 μL of bacterial lysate was transferred into the microwell containing the functionalized nanosensing substrate. For quantitative DNA samples, genomic material was diluted to desired concentration in desired sample matrix. Identical to above, 8 μL of bacterial lysate was transferred into the microwell containing the functionalized nanosensing substrate. All samples have been stored long-term at −20° C.

Spectral collection and plasmonic peak quantitation were conducted using Applicant's proprietary readout instrument and user interface. The optical readout instrument includes a spectrometer, light source, automated programmable stage (FIGS. 11A-11B). This hardware is coupled to a simple-to-operate user interface, which identifies resonance peak locations and calculates spectral shifts. Specifically, for each sample, a full transmission spectrum (500 nm-1000 nm) was collected for both the nanoarray and the glass slide background. The normalized transmittance spectrum was calculated as the ratio of the signal to background at every wavelength. The extinction was then calculated as the negative natural log of the normalized transmittance. These extinction spectra were smoothed using Lowess smoothing (10% smoothing) before the resonance peak wavelength was calculated. The resonance peak wavelength was determined through a center of mass calculation using numerical integration with wavelength bounds 700 nm to 900 nm. Spectral shifts were calculated by subtracting sample resonance peak locations from buffer resonance peak locations.

### Example 11: Nanoplasmonic Detection of Species-Specific Bacterial Genes and Antimicrobial Resistance Genes with PNA Probes

The performance of the PNA probes were assessed by determining their ability to bind their respective target sequences, as well as the probes specificity to the organism and/or gene of interest. The PNA probes were designed for UTI-causing pathogens. Genes that are conserved within the desired group of targeted pathogens, but distinct enough from their nearest neighbors were determined through literature survey and bioinformatic analyses for species identification. Reference genes available in the National Center for Biotechnology Information (NCBI) database were used as targets for AMR markers. Homologous (Identity>80%, Coverage>80%) sequence alignment records of target genes were then retrieved from the NCBI's nucleotide database using the Basic Local Alignment Tool (BLAST). The sequence alignments were used to identify potential locations for probe placement. Accordingly, oligonucleotide sequences (Table 12) that satisfy the required analytical inclusivity and specificity as evaluated against the NCBI's nucleotide and reference genome databases, were determined.

TABLE 12
Probe Sequences for Target Pathogens and Resistance Genes

| Channel/Probe Name | Sequence | Target |
|---|---|---|
| Enterococcus | ATACATGCAAGTCGAACGCTTCTTT | 16S rRNA |
| | (SEQ ID NO: 1) | |
| Escherichia coli | CCACAAACCGTTCTACTTTACTGGC | uidA |
| | (SEQ ID NO: 2) | |
| Klebsiella pneumoniae | TCACCCTGTTTGATGGCCGCACC | rpoB |
| | (SEQ ID NO: 3) | |
| Vancomycin-resistant Enterococci | CTGAGCTTTGAATATCGCAGCCT | vanA |
| | (SEQ ID NO: 4) | |
| ESBL-producing Escherichia coli | CGGTTCGCTTTCACTTTTCTTCAGC | blaCTX-M-1 |
| | (SEQ ID NO: 6) | |

The following thermodynamic criteria was used to design PNA probe for optimal performance.
Probe length: 15 to 30 nucleotides
Purine content: <50%
Melting temperature of the probe-target hybrid: 72° C. to 88° C.
Melting temperatures of monomeric secondary structures of the probe <50° C.

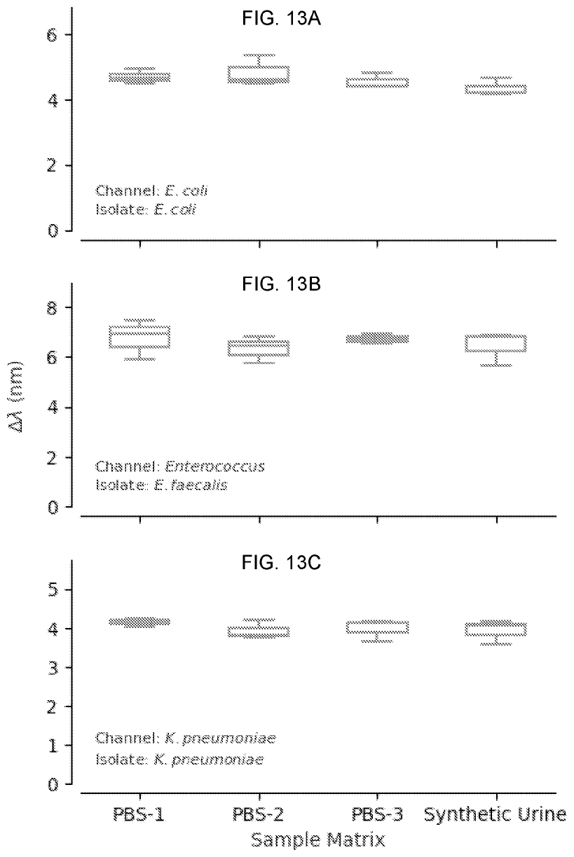

In order to determine if the PNA probes successfully bound target organisms, viable bacteria (E. coli, E. faecalis, and K. pneumoniae) were suspended in both phosphate buffered saline (PBS) and synthetic urine matrices, thermally lysed, and then exposed to the nanoplasmonic sensing substrate containing a PNA probe complimentary to the target sequence. For all three bacterial species, there was a significant red-shift in peak absorbance wavelength of the sensor, suggesting successful hybridization of the target nucleic acid sequence to the complementary PNA probe (FIGS. 13A-13C). The magnitude of peak shift ranged from 3.59 nm to 7.45 nm, for K. pneumoniae and E. faecalis, respectively. The average peak wavelength shift each organism in PBS was 4.02±0.07 nm, and 4.68±0.10 nm, and 6.61±0.17 nm for K. pneumoniae, E. coli, and E. faecalis, respectively. The average peak wavelength shift each organism in synthetic urine was 3.94±0.18 nm, and 4.35±0.15 nm, and 6.45±0.40 nm for K. pneumoniae, E. coli, and E. faecalis, respectively.

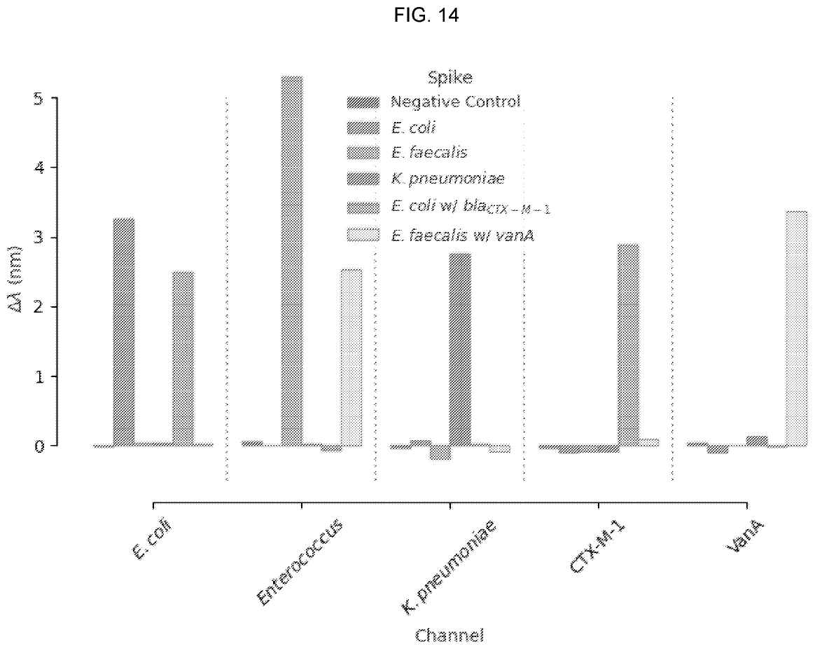

Next, probe specificity was characterized to target organism and/or resistance gene. As shown in FIG. 14, PNA probes, or target “channels” were observed to be highly specific to their target of interest. There was no significant cross-reactively observed between bacterial targets and selected target genes. Additionally, E. coli w/bla_{CTX-M-1} was detected via peak wavelength shift in both the E. coli and CTX-M-1 channel, but was not detected in any of the other three off-target channels.

### Example 12: Characterization of Nanosensor Limit-of-Detection for Five Targets in Synthetic Urine Matrix (3 UTI-Causing Pathogens; 2 Antimicrobial Resistance Genes)

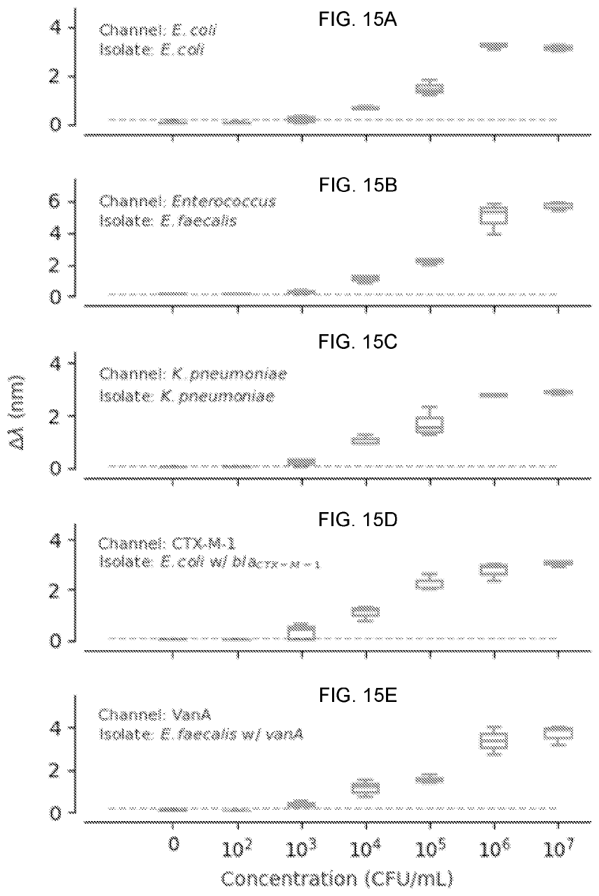

Following evaluation of the PNA probes for nanoplasmonic detection of species-specific bacterial and antimicrobial resistance genes, the limit-of-detection for nanoplasmonic molecular sensor was quantified for each of the five organism or resistance gene targets in a synthetic urine matrix (FIG. 15A-15E). The five target-panel was comprised of PNA probes designed for the specific detection of E. coli, Enterococcus spp., K. pneumoniae, CTX-M-1, & vanA. Limit-of-detection for each target (or channel) was quantified using Escherichia coli ATCC #25922 lysate, Enterococcus faecalis ATCC #29212 lysate, Klebsiella pneumoniae ATCC #13883 lysate, extended-spectrum beta-lactamase (ESBL)-producing Escherichia coli #BAA-2326 quantitative genomic DNA, and vancomycin-resistant Enterococcus faecium (VRE) #700221 quantitative genomic DNA, respectively.

For all five targets, a significant peak wavelength shift was first observed at a cell load (or equivalent) of approximately 10⁴ CFU/mL. The magnitude of the peak wavelength shift (i.e. signal) successively increased with increasing target concentration, suggesting the feasibility of semi-quantitative sample characterization. In case of UTIs, the ability to output a semi-quantitative result is necessary for effective clinical management. Current guidelines generally define a clinically significant UTI as ≥10⁵ CFU/mL. That said, the large majority of clinical microbiology laboratories in the United States will report culture results when 10³-10⁴ CFU/mL are detected; treatment decisions are then left to the discretion of the clinician. Although the current dynamic range the nanoplasmonic sensor disclosed herein is within relevant range to rule-in clinically significant UTIs, future efforts should work towards lowering the limit-of-detection to 103 CFU/mL in effort to capture the full spectrum of potentially clinically useful information.

### Example 13: Evaluation of Nanosensor Performance in Healthy Patient Urine Sample Matrix

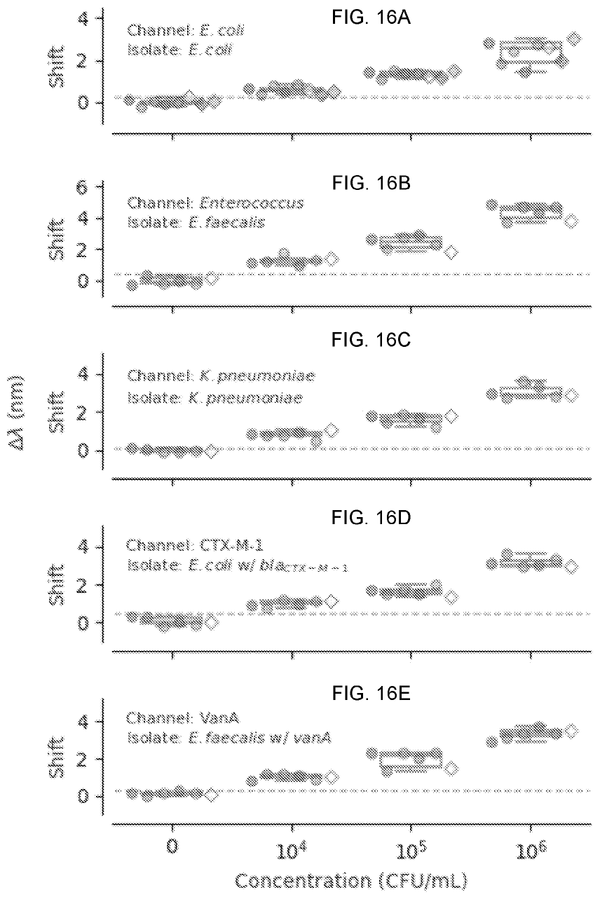

Target organisms and antimicrobial resistance genes were spiked into healthy patient urine samples to evaluate potential matrix effects (i.e. pH, salt concentration) on sensor performance. Midstream urine samples were collected from five patients and bacteria and/or genomic material was spiked into urine at known concentrations. Both individual patient urine sample matrices and pooled patient sample matrices were analyzed (FIGS. 16A-16E). Real patient urine sample matrices had no significant effect on the nanoplasmonic sensor performance. In all patient samples analyzed, target material was successfully detected in all five channels, limit-of-detection remained at 10⁴ CFU/mL, and sensor signal increased linearly with increasing target material. This set of experiments suggest that the nanoplasmonic platform can 1) rule-in clinically significant UTIs, 2) identify the UTI-causing organism, and 3) characterize key antimicrobial resistance profiles within 15 minutes. To the best of the Applicant's knowledge, this platform technology is enabling the first DNA-based test for UTI diagnosis and characterization at the point-of-care.

This platform can provide species-level information and key antibiotic susceptibility data without the need for nucleic acid amplification, effectively shortening time-to-diagnosis, decreasing cost, and limiting the need for external reagents. The technology platform described herein also has applications that extend beyond the detection of UTIs. Embodiment applications of this technology platform include the diagnosis of sexually transmitted infections, bloodstream infections, cancer screening, and biosecurity surveillance.

The scope of the present disclosure is not intended to be limited by the specific disclosures of examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A nanoplasmonic sensor comprising:
an array of functionalized sensors;
wherein each of the functionalized sensors in the array comprises an array of nanostructures conjugated to a biological probe; and
the biological probe is configured to detect the presence of an urinary tract infection-causing pathogen.

2. The nanoplasmonic sensor of claim 1, wherein the biological probe is a peptide nucleic acid probe or an oligonucleotide probe.

3. The nanoplasmonic sensor of claim 1, wherein at least one of the functionalized sensors in the array comprises a different biological probe for detecting a different urinary tract infection-causing pathogen from the other functionalized sensors.

4. The nanoplasmonic sensor of claim 3, wherein the nanoplasmonic sensor is configured to simultaneously detect multiple strands or species of the urinary tract infection-causing pathogens.

5. The nanoplasmonic sensor of claim 3, wherein each of the functionalized sensors in the array comprises a different biological probe.

6. The nanoplasmonic sensor of claim 1, wherein the urinary tract infection-causing pathogen is selected from the group consisting of Escherichia coli, Klebsiella pneumoniae, Proteus mirabilis, Enterococcus faecalis, Staphylococcus saprophyticus, and an antibiotic-resistant strain or identified resistance gene thereof.

7. The nanoplasmonic sensor of claim 1, wherein the biological probe has a sequence selected from the group consisting of SEQ ID NOS 1-32.

8. The nanoplasmonic sensor of claim 1, wherein the nanostructures comprise gold.

9. The nanoplasmonic sensor of claim 1, wherein the nanostructures in the array are regularly-spaced apart with a spacing of from about 100 nm and about 2000 nm, and each nanostructure has a square shape with a side dimension of from about 50 nm to about 400 nm.

10. The nanoplasmonic sensor of claim 9, wherein the nanostructures have a thickness of from about 20 nm to about 75 nm.

11. A nanoplasmonic sensor of claim 1, wherein a single biological probe can bind nucleic acids derived from more than one urinary tract infection-causing pathogens.

12. A method for detecting the presence of one or more urinary tract infection-causing pathogens comprising:
exposing the nanoplasmonic sensor of claim 1 to a bodily fluid sample of a patient suspecting of having urinary tract infection;
illuminating a light at a series of wavelengths onto each of the functionalized sensors; and
collecting absorbance, transmittance, or extinction data of each of the functionalized sensors.

13. The method of claim 12, further comprising heating the nanoplasmonic sensor after exposing the nanoplasmonic sensor to the bodily fluid sample.

14. The method of claim 12, further comprises comparing the collected absorbance, transmittance, or extinction data of each functionalized sensor with a baseline data of each of the functionalized sensor prior to exposure to the bodily fluid sample.

15. The method of claim 14, wherein the comparing step reveals an optical peak shift when a urinary tract infection-causing pathogen is detected.

16. The method of claim 15, wherein the amount of the optical peak shift is correlated to the concentration of the urinary tract infection-causing pathogen in the bodily fluid sample.

17. The method of claim 12, wherein the bodily fluid sample comprises urine, saliva, blood, plasma, serum, or mucus.

18. The method of claim 12, wherein at least one of the functionalized sensors in the array comprises a different biological probe for detecting a different urinary tract infection-causing pathogen from the other functionalized sensors.

19. The method of claim 18, wherein the urinary tract infection-causing pathogen is independently selected from the group consisting of Escherichia coli, Klebsiella pneumoniae, Proteus mirabilis, Enterococcus faecalis, Staphylococcus saprophyticus, and an antibiotic-resistant strain or identified resistance gene thereof.

20. The method of claim 18, wherein multiple strains or species of the urinary tract infection-causing pathogens are detected simultaneously.

21. The method of claim 12, wherein the biological probe has a sequence independently selected from the group consisting of SEQ ID NOS: 1-32.

22. The method of claim 12, wherein each of the functionalized sensors in the array comprises a different biological probe.

23. The method of claim 12, wherein the method is configured to be performed at the point of care.

24. A method for detecting the presence of one or more urinary tract infection-causing pathogens, comprising:
providing a sensor comprising one or more biological probes designed to detect one or more target nucleic acid sequences derived from one or more urinary tract infection-causing pathogens;
exposing the sensor to a sample that is suspected to contain one or more urinary tract infection-causing pathogens; and
collecting electrical, fluorescent, absorbance, transmittance, and/or extinction data from the sensor.

25. The method of claim 24 wherein the one or more biological probes were selected using computational and/or bioinformatic methods.

26. The method of claim 24 wherein the one or more biological probes contain intentionally varying degrees of mismatch with the one or more target nucleic acids sequences.

27. The method of claim 24 wherein the one or more biological probes are designed to bind multiple target nucleic acid sequences.

28. The method of claim 24 wherein one of the biological probes can bind nucleic acids derived from more than one urinary tract infection-causing pathogen.

29. The method of claim 24 wherein the one or more biological probes are designed to bind nucleic acid sequences specific to antibiotic resistance genes.

30. The method of claim 24 wherein one of the biological probes can bind nucleic acid sequences from more than one antibiotic resistance genes.

31. The method of claim 24, wherein the one or more biological probes have sequences that are independently selected from the group consisting of SEQ ID Nos. 1-32.
